SEQUENCE LISTING (2 sequences)

SEQ ID NO: 1 - protein, 24 aa, Artificial Sequence
  peptide fragment of Human Histone H3
ARTKQTARKSTGGKAPRKQLAGGK

SEQ ID NO: 2 - protein, 14 aa, Artificial Sequence
  Woodtide peptide fragment
KKISGRLSPIMTEQ

(19) United States
(12) Patent Application Publication
(10) Pub. No.: US 2013/0102627 A1
(43) Pub. Date: Apr. 25, 2013

(54) Acridines As Inhibitors Of Haspin And DYRK Kinases

(71) Applicants: Jonathan Higgins, Chestnut Hill, MA (US); Gregory D. Cuny, Cambridge, MA (US); Marcie Glicksman, Winchester, MA (US); Debasis Patnaik, Quincy, MA (US); Maxime Robin, Marseille (FR); Ross L. Stein, Cambridge, MA (US); Jun Xian, Sharon, MA (US)

(72) Inventors: Jonathan Higgins, Chestnut Hill, MA (US); Gregory D. Cuny, Cambridge, MA (US); Marcie Glicksman, Winchester, MA (US); Debasis Patnaik, Quincy, MA (US); Maxime Robin, Marseille (FR); Ross L. Stein, Cambridge, MA (US); Jun Xian, Sharon, MA (US)

(73) Assignee: THE BRIGHAM AND WOMEN'S HOSPITAL, INC., Boston, MA (US)

(21) Appl. No.: 13/639,569

(22) PCT Filed: Apr. 8, 2011
(86) PCT No.: PCT/US11/31786

§ 371 Date: Dec. 13, 2012

Related U.S. Application Data
(60) Provisional application No. 61/322,580, filed on Apr. 9, 2010.

(51) Int. Cl. C07D 401/12; C07D 219/12; C07D 219/06

(52) U.S. Cl. CPC C07D 401/12; C07D 219/06; C07D 219/12
USPC 514/297; 546/103; 546/106; 546/104

(57) ABSTRACT

The present disclosure is directed to compounds of Formula I: which are inhibitors of Haspin kinase and DYRK kinases. The compounds of the present disclosure, and compositions thereof, are useful in the treatment of disease related to Haspin kinase and DYRK kinase expression and/or activity.

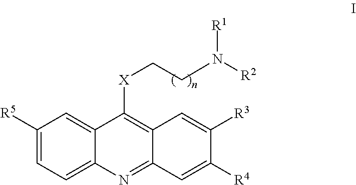

### CROSS REFERENCE TO RELATED APPLICATION

This application claims priority to U.S. Provisional Patent Application No. 61/322,580, filed on Apr. 9, 2010, which is incorporated herein by reference in its entirety.

### STATEMENT REGARDING FEDERALLY SPONSORED RESEARCH

This invention was made with Government support under National Institutes of Health Grants No. R01CA122608. The Government has certain rights in this invention.

### TECHNICAL FIELD

The present disclosure relates to acridine compounds that inhibit the activity of kinases such as Haspin and DYRKs. In some embodiments, the compounds are selective for Haspin and/or DYRK2. The compounds can be used, for example, to treat diseases associated with kinase expression or activity such as cancer.

### BACKGROUND

Haspin (Haploid Germ Cell-Specific Nuclear Protein Kinase), also known as Gsg2 (Germ Cell Specific Gene-2) (Tanaka, H. et al. J. Biol. Chem. 274:17049, 1999; Tanaka, H. et al. FEBS Lett. 355:4, 1994), is a serine/threonine kinase expressed in a variety of tissues (e.g. testis, bone narrow, thymus and spleen) and in proliferating cells. Haspin's kinase activity functions during mitosis, where it has been shown to phosphorylate histone H3 at Thr-3 (H3T3). Depletion of haspin by RNA interference significantly reduces H3 Thr-3 phosphorylation in cells and prevents normal completion of mitosis.

DYRKs (Dual-specificity Tyrosine-regulated Kinases) belong to the CMGC family of ePKs and contain a conserved kinase domain and adjacent N-terminal DYRK homology box. This group of kinases can be further divided into class 1 kinases (DYRK1A and 1B) that have an N-terminal nuclear localization signal and a C-terminal PEST region and class 2 kinases (DYRK2, 3 and 4), which lack these motifs and are predominantly cytosolic. Although DYRKs phosphorylate substrates on serine or threonine residues, their activity depends upon autophosphorylation of an essential activation loop tyrosine during synthesis (Lochhead, P. A. et al. Cell 121: 925, 2005). DYRK kinases appear to contribute to regulation of an array of signaling pathways, including NFAT signaling in the brain and immune system, Hedgehog signaling, caspase activity during apoptosis, cell cycle progression and mitosis, and p53 activation in response to DNA damage.

The identification of compounds that inhibit the activity of Haspin and/or DYRKs represents a desirable drug design approach for the needed development of pharmacological agents for the treatment of diseases associated with Haspin and DYRK activity. The compounds described herein help fulfill these and other needs.

### SUMMARY

The present disclosure provides compounds of Formula I:

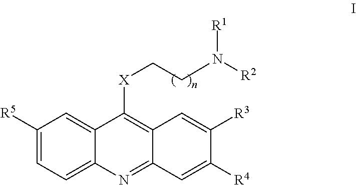

or pharmaceutically acceptable salts thereof, wherein the constituent members are provided herein.

The present disclosure further provides compositions comprising a compound of Formula I and a pharmaceutically acceptable carrier and a pharmaceutically acceptable salt thereof.

The present disclosure further provides methods of treating a disease in a subject by administering to the subject a therapeutically effective amount of a compound of Formula I, or pharmaceutically acceptable salt thereof. In some embodiments, the disease is cancer, Down's Syndrome, diabetes, cardiac ischemia, Alzheimer's Disease, or anemia.

In some embodiments, the disease is cancer, e.g. a hematological malignancy, e.g. leukemia or lymphoma

The present disclosure further provides compounds of Formula I, or pharmaceutically acceptable salts thereof, for use in therapy.

The present disclosure further provides compound of Formula I, or pharmaceutically acceptable salts thereof, for use in the preparation of a medicament for use in therapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1, 2A and 2B depict graphs of dose response curves for compound LDN-192960.

FIG. 3A and 3B are tables of in vitro testing results for compound LDN-192960 against representative NCI cell lines.

FIG. 4A and 4B are graphs depicting mean values for a dose titration of compound LDN-192960.

FIG. 5A and 5B are graphs depicting mean values for single dose titration of compound LDN-192960.

### DETAILED DESCRIPTION

The present disclosure provides, inter alia, compounds that are inhibitors of kinases, including serine/threonine kinases such as Haspin, and those of the CMGC family of eukaryotic protein kinase (ePK) such as DYRK2, having Formula I:

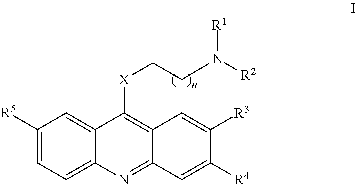

or pharmaceutically acceptable salt thereof, wherein:

X is CH₂, S, or NR^{A};

R¹ and R² are each independently H, C₁₋₆alkyl, —C(O)R^{A}, —C(O)OR^{A}, or —C(O)NR^{A}R^{B};

or R¹ and R² together with the N atom to which they are attached form a heterocyclic group selected from: a phthalimide group, a benzo[d]isothiazol-3(2H)-one 1,1-dioxide, a benzo[d][1,3,2]dithiazole 1,1,3,3-tetraoxide, a 3-iminoisoindolin-1-one, and an isoindoline-1,3-diimine; wherein the phthalamide group is optionally substituted by halo, OH, C₁₋₆haloalkyl, C₁₋₆alkoxy, C₁₋₆haloalkoxy, —CN, —C(O)NR^{A}R^{B}, —S(O)₂R^{A}, —S(O)₂NR^{A}R^{B}, or —NR^{A}R^{B};

R³, R⁴ and R⁵ are each independently H, halo, OH, C₁₋₆haloalkyl, C₁₋₆alkoxy, C₁₋₆haloalkoxy, —CN, —C(O)NR^{A}R^{B}, —S(O)₂R^{A}, —S(O)₂NR^{A}R^{B}, or —NR^{A}R^{B};

R^{A} and R^{B} are each independently H or C₁₋₆alkyl; and

n is 1, 2, 3, 4, or 5.

In some embodiments, X is S or CH₂.

In some embodiments, R¹ and R² are each independently H, C₁₋₆alkyl, or R¹ and R² together with the N atom to which they are attached form a phthalimide group, optionally substituted by halo, OH, C₁₋₆haloalkyl, C₁₋₆alkoxy, C₁₋₆haloalkoxy, —CN, —C(O)NR^{A}R^{B}, —S(O)₂R^{A}, —S(O)₂NR^{A}R^{B}, or —NR^{A}R^{B}.

In some embodiments, X is S, and R¹ and R² are both H.

In some embodiments, R³ and R⁵ are both C₁₋₆alkoxy.

In some embodiments, R⁴ is halo.

In some embodiments, R⁴ is chloro.

In some embodiments, n is 2.

In some embodiments, the compound has Formula I, wherein:

X is S or CH₂;

R¹ and R² are each H;

R³ and R⁵ are each independently H, OH, methyl, methoxy, or chloro; and

n is 2 or 3.

In some embodiments, the compound has Formula I, wherein:

X is S;

R¹ and R² are each independently H or methyl;

R³ and R⁵ are each independently H or methoxy; and

n is 2 or 3.

In some embodiments, the compound has Formula I, wherein:

X is S;

R¹ and R² are each H, or R¹ and R² together with the N atom to which they are attached form an unsubstituted phthalimide group;

R³ is methoxy;

R⁴ is H;

R⁵ is methoxy; and

n is 2.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further intended that the compounds described herein are stable. As used herein “stable” refers to a compound that is sufficiently robust to survive isolation to a useful degree of purity from a reaction mixture, and preferably capable of formulation into an efficacious therapeutic agent.

It is further appreciated that certain features of the present disclosure, which are, for clarity, described in the context of separate embodiments, can also be provided in combination in a single embodiment. Conversely, various features of the present disclosure which are, for brevity, described in the context of a single embodiment, can also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkoxy” refers to an —O-alkyl group. Example alkoxy groups include methoxy, ethoxy, propoxy (e.g., n-propoxy and isopropoxy), t-butoxy, and the like.

As used herein, “halo” or “halogen” includes fluoro, chloro, bromo, and iodo.

As used herein, “phthalamide” refers to

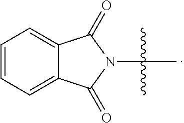 [SMILES: *N1C(=O)c2ccccc2C1=O ; "."]

As used herein, “benzo[d]isothiazol-3(2H)-one 1,1-dioxide” refers to

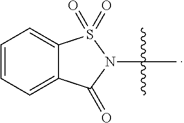 [SMILES: *N1C(=O)c2ccccc2S1(=O)=O ; "."]

As used herein, “benzo[d][1,3,2]dithiazole 1,1,3,3-tetraoxide” refers to

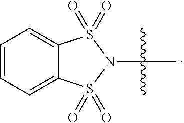 [SMILES: *N1S(=O)(=O)c2ccccc2S1(=O)=O ; "."]

As used herein, “3-iminoisoindolin-1-one” refers to

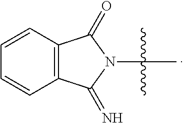 [SMILES: *N1C(=N)c2ccccc2C1=O ; "."]

As used herein, “isoindoline-1,3-diimine” refers to

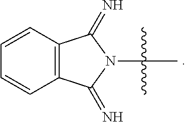 [SMILES: *N1C(=N)c2ccccc2C1=N]

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Compounds of the present disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, amide-imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure can also include all isotopes of atoms occurring in the intermediates or final compounds. Isotopes include those atoms having the same atomic number but different mass numbers. For example, isotopes of hydrogen include tritium and deuterium.

In some embodiments, the compounds of the present disclosure, and salts thereof, are substantially isolated. By “substantially isolated” is meant that the compound is at least partially or substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Synthesis

The novel compounds of the present disclosure can be prepared in a variety of ways known to one skilled in the art of organic synthesis. The compounds of the present disclosure can be synthesized using the methods as hereinafter described below, together with synthetic methods known in the art of synthetic organic chemistry or variations thereon as appreciated by those skilled in the art.

The compounds of described herein can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry (e.g., liquid chromatography-mass spectrometry (LC-MS)), or by chromatography such as high performance liquid chromatography (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Green's Protective Groups in Organic Synthesis, 4d. Ed., Wiley & Sons, 2006, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

The compounds of the present disclosure can be prepared, for example, using the reaction pathways and techniques as described in the Examples below.

### Methods of Use

Compounds of the present disclosure can modulate activity of protein kinases. Example protein kinases modulated by the compounds of the present disclosure include serine/threonine kinases. In some embodiments, the kinase is a member of the CMGC family of eukaryotic protein kinases (ePKs). In some embodiments, the compounds described herein inhibit activity of Haspin kinase. In some embodiments, the compounds described herein inhibit Dual-specificity Tyrosine-regulated Kinases (DYRKs), e.g. DYRK2.

In some embodiments, the compounds of the present disclosure inhibit phosphorylation of histone H3 at a Thr-3 by Haspin. Thus, the present disclosure further provides methods of inhibiting a ligand/kinase signaling pathway such as the Haspin kinase signaling pathway in a cell by contacting the cell with a compound of the present disclosure. The present disclosure further provides methods of inhibiting proliferative activity of a cell by contacting the cell with a compound described herein.

The present disclosure further provides methods of treating diseases associated with a dysregulated kinase signaling pathway, including abnormal activity and/or overexpression of the protein kinase, in a subject (e.g., human) by administering to the subject in need of such treatment a therapeutically effective amount or dose of a compound of the present disclosure or a pharmaceutical composition thereof. In some embodiments, the dysregulated kinase is a serine/threonine kinase (e.g., Haspin or DYRKs). In some embodiments, the dysregulated kinase is overexpressed in the diseased tissue of the subject. In some embodiments, the dysregulated kinase is abnormally active in the diseased tissue of the subject. In some embodiments, the dysregulated kinase is a kinase that is associated with the Haspin/DYRK pathway.

In some embodiments, the compounds of the present disclosure are useful in treating diseases such as cancer, Down's Syndrome, diabetes, cardiac ischemia, Alzheimer's Disease, anemia, or arthritis. In some embodiments, the compounds can be used as a therapeutic approach in Down's Syndrome (DYRK1A) (Anon, J. R. et al. Nature 441: 595-600, 2006; Laguna, A. et al. Developmental cell 15: 841-853, 2008; Kim, N. D. Bioorganic & medicinal chemistry letters 16: 3772-3776, 2006; Ortiz-Abalia, J. et al. American journal of human genetics 83: 479-488, 2008, each of which is incorporated herein by reference in its entirety).

In some embodiments, the compounds of the present disclosure can inhibit DYRK1A or DYRK2 by activating NFAT, and therefore, may have immunomodulatory features of benefit in immune-compromised states, or may increase pancreatic β-cell function in diabetes (Gwack, Y. et al. Nature 441: 646-650, 2006; Heit, J. J., et al. Nature 443: 345-349, 2006; Heit, J. J. Bioessays 29: 1011-1021, 2007, each of which is incorporated herein by reference in its entirety).

In some embodiments, the compounds of the present disclosure can be useful for stimulating blood vessel growth following cardiac ischemia (Varjosalo, M., et al. Cell 133: 537-548, 2008), or for treating neurological conditions such as Alzheimer's disease (Briscoe, J. et al. Nature chemical biology 2: 10-11, 2006; Longo, F. M. et al. Current Alzheimer research 3: 5-10, 2006, each of which is incorporated herein by reference in its entirety).

In some embodiments, the compounds of the present disclosure can be useful as anti-anemia agents (Bogacheva, O., et al. The Journal of Biological Chemistry 283:

36665-36675, 2008; Lord, K. A., et al. Blood 95: 2838-2846, 2000; Geiger, J. N., et al. Blood 97: 901-910, 2001, each of which is incorporated herein by reference in its entirety).

In some embodiments, the compounds of the present disclosure can be useful for in vitro programming of cell fate to obtain cells for regenerative therapy that may circumvent some of the problems inherent in genetic manipulation of cells and the side effects of drugs in patients (Emre, N., et al. Curr Opin Chem Biol 11: 252-258, 2007; Borowiak, M., et al. Curr Opin Cell Biol 21: 727-732, 2009, each of which is incorporated herein by reference in its entirety).

In some embodiments, the compounds of the present disclosure are useful in treating diseases such as cancer. In further embodiments, the compounds of the present disclosure can be useful in methods of inhibiting tumor growth or metastasis of a tumor in a subject.

Example cancers treatable by the methods herein include is leukemia, e.g., acute lymphoblastic leukemia, acute myelogenous leukemia, chronic lymphocytic leukemia, chronic myelogenous leukemia, acute monocytic leukemia; lymphoma, e.g. Hodgkin's lymphoma, non-Hodgkin's lymphoma, B cell or T cell lymphoma, or T cell leukemia; myeloma, e.g. multiple myeloma, and the like.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a compound of the present disclosure with a protein kinase includes the administration of a compound of the present disclosure to an individual or patient, such as a human as well as, for example, introducing a compound of the present disclosure into a sample containing a cellular or purified preparation of the protein kinase.

As used herein, the term “subject” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician, which includes one or more of the following:

(1) reducing the risk of developing the disease; for example, reducing the risk of developing a disease, e.g. cancer, condition or disorder in an individual who may be predisposed to the disease, e.g. cancer, condition or disorder but does not yet experience or display the pathology or symptomatology of the disease, e.g. cancer;

(2) inhibiting the disease; for example, inhibiting a disease, condition or disorder in an individual who is experiencing or displaying the pathology or symptomatology of the disease, e.g. cancer, condition or disorder; and

(3) ameliorating the disease; for example, ameliorating a disease, e.g. cancer, condition or disorder in an individual who is experiencing or displaying the pathology or symptomatology of the disease, e.g. cancer, condition or disorder (i.e., reversing the pathology and/or symptomatology) such as decreasing the severity of disease, e.g. cancer.

### Combination Therapy

One or more additional pharmaceutical agents or treatment methods such as, for example, chemotherapeutics, anti-cancer agents, cytotoxic agents, or anti-cancer therapies (e.g., radiation, hormone, etc.), can be used in combination with the compounds of the present disclosure for treatment of the diseases, disorders or conditions described herein. The agents or therapies can be administered together with the compounds of the present disclosure (e.g., combined into a single dosage form), or the agents or therapies can be administered simultaneously or sequentially by separate routes of administration.

Suitable anti-cancer agents include kinase inhibiting agents including trastuzumab (Herceptin), imatinib (Gleevec), gefitinib (Iressa), erlotinib hydrochloride (Tarceva), cetuximab (Erbitux), bevacizumab (Avastin), sorafenib (Nexavar), sunitinib (Sutent), and RTK inhibitors described in, for example, WO 2005/004808, WO 2005/004607, WO 2005/005378, WO 2004/076412, WO 2005/121125, WO 2005/039586, WO 2005/028475, WO 2005/040345, WO 2005/039586, WO 2003/097641, WO 2003/087026, WO 2005/040154, WO 2005/030140, WO 2006/014325, WO 2005/070891, WO 2005/073224, WO 2005/113494, and US Pat. App. Pub. Nos. 2005/0085473, 2006/0046991, and 2005/0075340.

Suitable chemotherapeutic or other anti-cancer agents further include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Suitable chemotherapeutic or other anti-cancer agents further include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol™), mithramycin, deoxyco-formycin, mitomycin-C, L-asparaginase, interferons (especially IFN-a), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as antibodies to costimulatory molecules such as CTLA-4, 4-1BB and PD-1, or antibodies to cytokines (IL-10, TGF-β, etc.). Further antibody therapeutics include antibodies to serine/threonine kinases and/or their ligands such as anti-Haspin antibodies and/or anti-DYRK antibodies. The term “antibody” is meant to include whole antibodies (e.g., monoclonal, polyclonal, chimeric, humanized, human, etc.) as well as antigen-binding fragments thereof.

Other anti-cancer agents also include those that augment the immune system such as adjuvants or adoptive T cell transfer.

Other anti-cancer agents include anti-cancer vaccines such as dendritic cells, synthetic peptides, DNA vaccines and recombinant viruses.

Other anti-cancer agents include Aurora B inhibitors and Aurora A, Plk1, and kinesin-5 inhibitors (Lens, S. M., et al. Nat. Rev. Cancer 10: 825-841, 2010).

Methods for the safe and effective administration of most of the above agents are known to those skilled in the art. In addition, their administration is described in the standard literature. For example, the administration of many of the chemotherapeutic agents is described in the “Physicians' Desk Reference” (PDR, e.g., 2011 edition, PDR Network), the disclosure of which is incorporated herein by reference as if set forth in its entirety.

### Pharmaceutical Formulations and Dosage Forms

When employed as pharmaceuticals, the compounds of the present disclosure can be administered in the form of pharmaceutical compositions which is a combination of a compound of the present disclosure and a pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The present disclosure also includes pharmaceutical compositions which contain, as the active ingredient, one or more of the compounds of the present disclosure in combination with one or more pharmaceutically acceptable carriers. In making the compositions of the present disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the present disclosure can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present disclosure can vary according to, for example, the particular use for which the treatment is made, the manner of administration of the compound, the health and condition of the patient, and the judgment of the prescribing physician. The proportion or concentration of a compound of the present disclosure in a pharmaceutical composition can vary depending upon a number of factors including dosage, chemical characteristics (e.g., hydrophobicity), and the route of administration. For example, the compounds of the present disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the present disclosure can also be formulated in combination with one or more additional active ingredients which can include any pharmaceutical agent such as anti-viral agents, vaccines, antibodies, immune enhancers, immune suppressants, anti-inflammatory agents and the like.

### Labeled Compounds and Assay Methods

Another aspect of the present disclosure relates to fluorescent dye, spin label, heavy metal or radio-labeled compounds of the present disclosure that would be useful not only in imaging but also in assays, both in vitro and in vivo, for localizing and quantitating the protein kinase target in samples, e.g. samples comprising cells or tissues, including human, and for identifying kinase ligands by inhibition of binding of a labeled compound. Accordingly, the present disclosure includes kinase enzyme assays that contain such labeled compounds.

The present disclosure further includes isotopically-labeled compounds of the compounds described herein. An “isotopically” or “radio-labeled” compound is a compound of the present disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro IDO enzyme labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for incorporating radio-isotopes into organic compounds are applicable to compounds of the present disclosure and are well known in the art.

A radio-labeled compound of the present disclosure can be used in a screening assay to identify/evaluate compounds. In general terms, a newly synthesized or identified compound (i.e., test compound) can be evaluated for its ability to reduce binding of the radio-labeled compound of the present disclosure to the enzyme. Accordingly, the ability of a test compound to compete with the radio-labeled compound for binding to the enzyme directly correlates to its binding affinity.

### Kits

The present disclosure also includes pharmaceutical kits useful, for example, in the treatment or prevention of diseases, such as cancer and other diseases referred to herein, which include one or more containers containing a pharmaceutical composition comprising a therapeutically effective amount of a compound of the present disclosure, or pharmaceutically acceptable salt thereof. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The present disclosure will be described in greater detail by way of specific examples. The following examples are offered for illustrative purposes, and are not intended to limit the present disclosure in any manner. Those of skill in the art will readily recognize a variety of noncritical parameters which can be changed or modified to yield essentially the same results. The compounds of the Examples were found to be inhibitors of Haspin and/or DYRKs according to one or more of the assays provided herein.

### EXAMPLES

The invention is further described in the following examples, which do not limit the scope of the invention described in the claims.

### Example 1

### Expression and Purification of Recombinant Haspin

A synthetic codon-optimized human Haspin cDNA was made in vector pUC57 at GenScript Corporation (Piscataway, N.J.) to facilitate bacterial expression. This full-length Haspin gene was cloned into the pMALc2E vector (New England Biolabs, Ipswich, Mass.) using EcoR I and Sal I sites. Haspin was expressed and purified as an N-terminal MBP fusion protein from E. coli Rosetta™2(DE3)pLysS cells (Novagen, Madison, Wis.). A freshly transformed colony was used to initiate a small volume liquid culture in LB medium with 2 g/l glucose, 34 μg/ml chloramphenicol and 100 μg/ml ampicillin. This culture was used to inoculate a large volume of the same medium and grown until an absorbance at 600 nm of 0.5 was reached. Protein expression was induced by adding 0.3 mM isopropyl thiogalactoside and growth with shaking at room temperature for 14 hours. Affinity column chromatography was carried out using amylose resin following the manufacturer's instructions (New England Biolabs). The fusion protein was eluted in 50 mM Tris, pH 7.5, 200 mM NaCl, 10 mM maltose and dialyzed into 50 mM Tris, pH 7.5, 200 mM NaCl, 2 mM DTT and 50% glycerol. The purity and yield of intact fusion protein was determined by SDS-PAGE and Coomassie Blue staining, in comparison with known quantities of bovine serum albumin.

### Example 2

### Reagents and Substrates For Haspin Assay

A synthetic peptide representing the first 21 amino acid residues of human Histone H3 was designated H3(1-21)-biotin peptide (ARTKQTARKSTGGKAPRKQLA-GGK-biotin (SEQ ID No:1)) was synthesized at Abgent (San Diego, Calif.). This peptide carried biotin on the side chain of the C-terminal lysine. Recombinant full-length human histone H3 was obtained from New England Biolabs. ATP and Staurosporine were purchased from Sigma-Aldrich (St Louis, Mo.). Rabbit monoclonal anti-Histone H3T3ph antibody (clone JY325) from Millipore (Billerica, Mass.) was directly labeled by PerkinElmer (Waltham, Mass.) with LANCE Eu W1024. For indirect detection, LANCE Eu W1024 labeled anti-rabbit IgG antibody was used (PerkinElmer). Streptavidin conjugated to SureLight-Allophycocyanin (PerkinElmer) was used as the acceptor fluorophore.

### Example 3

### TR-FRET Haspin Assay

To identify Haspin inhibitors by high throughput screening, a homogeneous kinase assay based on time-resolved fluorescence resonance energy transfer (TR-FRET) was designed. Mathis described the application of TR-FRET to assay kinase activity (Mathis, G. et al. Clin. Chem. 41: 1391-1397, 1995), which has emerged as one of the preferred fluorescent assay formats in drug discovery. Such TR-FRET assays use a lanthanide donor species conjugated to a phospho-specific antibody that binds specifically to the product of kinase reaction labeled with an acceptor fluorophore. This induced proximity of the donor and acceptor fluorophores leads to resonance energy transfer, resulting in a detectable increase of TR-FRET signal.

In the assay described below, a Europium chelate, conjugated to an anti-Histone H3T3ph antibody, as the donor species was used. The acceptor fluorophore, allophycocyanin (APC) was used as a streptavidin conjugate that could bind to a biotinylated Histone H3 peptide substrate. The TR-FRET read-out is a dimensionless number calculated as a ratio of acceptor specific fluorescence signal to the donor signal, which provided a robust internal standard to compensate for compound interference and variations in assay volume (Hemmilä, I. J Biomol Screen 4: 303-308, 1999; Mathis, G. J Biomol Screen 4: 309-314, 1999). Lanthanide ions like Europium have a much longer emission lifetime, often measured in hundreds of microseconds, compared with traditional organic reagents that have lifetimes measured on the scale of hundreds of nanoseconds. TR-FRET assays are thus less susceptible to compound interference generated by short-lived compound or matrix component fluorescence. Furthermore, TR-FRET can be carried out in a homogeneous format that avoids time-consuming separation steps that introduce variability. Based on these properties TR-FRET based assay kinases have been widely used in high throughput screening.

The TR-FRET assay was utilized to screen a small molecule library of approximately 140000 compounds. Primary hits were re-tested by TR-FRET assay using the peptide substrate and then revalidated by assaying the compounds in a radiometric assay using full-length Histone H3 as a protein substrate. Candidate compounds were confirmed in a cellular assay of Haspin activity (Patnaik et al. J. Biomol. Screen. 13: 1025-1034, 2008, which is incorporated by reference in its entirety).

Utilizing the aforementioned assay compound LDN-192960 was identified as a Haspin kinase inhibitor. Furthermore, analogs were also prepared that also demonstrated Haspin kinase inhibitory activity. Some analogs were also found to be DYRK2 inhibitors. Inhibitory activity of compounds against Haspin and DYRK2 kinases are shown in Table 2, below.

A CRS CataLyst Express robotic arm (Thermo Fisher Scientific, Waltham, Mass.) and a Cybi-well 384 channel simultaneous pipettor (CyBio AG, Jena, Germany) were used to carry out the high throughput screening of a small molecule library. Kinase reactions were performed in 50 mM Tris, pH 7.5, 5 mM MgCl₂, 1 mM DTT, 0.01% Brij-35 using Proxiplate 384 Plus white assay plates (PerkinElmer). In the final HTS conditions, 0.17 nM enzyme (0.05 nM MBP-Haspin final) and 0.33 μM biotinylated H3(1-21) peptide (0.1 μM peptide final, at the Kₘ) in a volume of 3 μl kinase buffer were added to 2 μl solutions of compound (10 μM final for screening purposes) and pre-incubated for 20 minutes. The kinase reaction was initiated by addition of 5 μl of 400 μM ATP per reaction (200 μM ATP final, at the Kₘ). The reaction was incubated for 10 minutes at room temperature. Reaction was terminated by the addition of 10 μl 50 mM EDTA, 2 nM Europium labeled anti-Histone H3T3ph antibody, 40 nM Streptavidin-APC. After a two hour incubation at room temperature, TR-FRET measurements were performed using a PHERAstar HTS microplate reader (BMG Labtech, Offenberg, Germany), and were expressed as ratios of acceptor fluorescence at 665 nm over donor fluorescence at 620 nm.

### Example 4

### Radiometric Haspin Filter Binding Assay

In radiometric assays, 10 μM test compound was incubated with 4 nM MBP-Haspin in a 25 μl enzyme reaction containing 0.3 μM Histone H3 (slightly above the apparent Kₘ value of 0.18 μM for Histone H3 in this assay) and 11 μM ATP (apparent Kₘ value), 0.73 μCi γ³³P-ATP (PerkinElmer), 50 mM Tris-HCl, 5 mM MgCl₂, pH 7.5. The reaction was stopped after 10 minutes by directly spotting 10 μl of reaction mix on P81 phosphocellulose filters (Whatman plc, Maidstone, UK). P81 filter discs were subsequently washed thrice with 0.2 M ammonium bicarbonate (5 ml/circle) and air dried. The dried P81 filter discs were transferred to a 6 ml scintillation vial (Pony-Vial, PerkinElmer) and, following addition of 3 ml of scintillation fluid, were read using an LS5801 liquid scintillation counter (Beckman Coulter, Fullerton, Calif.). Background ³³P incorporation was defined from similar reactions carried out in the absence of enzyme.

### Example 5

### Cell Based Haspin ELISA Assay

For cell-based ELISA, myc-Haspin overexpressing HeLa Tet-on cells (Dai J et al. Genes and Development 19: 472-488, 2005, which is incorporated by reference in its entirety) were maintained in Dulbecco's modified Eagle medium (DMEM) supplemented with 10% Tet-system approved fetal bovine serum (Clontech, Mountain View, Calif.). Approximately 15,000 cells per well were seeded in a 96 well Nunclon™ Δ surface plates (Thermo Fisher Scientific). Following 16 hours growth in the presence of 1 μM doxycycline to induce myc-Haspin expression, cells were treated for 2 hours with various inhibitor concentrations. The cells were then fixed with 4% formaldehyde in PBS for 2 hours, followed by washing thrice with 200 μl Wash Buffer per well (PBS, 0.1% Triton X-100, pH 7.4). The wells of assay plate were subsequently treated with quench buffer (0.1% NaN₃, 1% H₂O₂ in Wash Buffer) for 1 hour. Then the plates were again washed thrice with Wash Buffer and incubated overnight at 4° C. with rabbit anti-Histone H3T3ph affinity-purified polyclonal antibody B8634 (Dai J et al. Genes and Development 19: 472-488, 2005, which is incorporated by reference in its entirety) in 3% BSA in Wash Buffer. The plates were warmed to room temperature, washed thrice with Wash Buffer and incubated with 1:3000 anti-rabbit IgG-HRP (Jackson Immunoresearch, West Grove, Pa.) in Wash Buffer for 1 hour. After washing thrice with Wash Buffer, a 1:1 mix of chemiluminescent substrate and hydrogen peroxide was added to each well (SuperSignal ELISA Pico Chemiluminescent Substrate, Thermo Fisher Scientific). Chemiluminescence was measured after five minute incubation on a Victor² Plate Reader (PerkinElmer). To control for cell viability, duplicate plates were assayed using CellTiter-Glo® (Promega, Madison, Wis.), following the manufacturer's protocol, which uses luciferase to measure ATP as an indicator of metabolically active viable cells.

### Example 6

### Data Analysis

Data were analyzed using GraphPad Prism Version 4 (GraphPad Software Inc, La Jolla, Calif.). No inhibitor (“MAX”) and Staurosporine inhibitor (“MIN”) controls were used to calculate Z′ values and signal to background ratios during the high throughput screen. Percentage inhibition of enzyme activity was calculated according to the following equation: % inhibition=100×(average of MAX controls—test compound value)/(average of MAX controls−average of MIN controls). For determination of IC₅₀ and EC₅₀ concentrations, mean % inhibition dose response curves were fitted to the sigmoidal dose response equation: Y=Bottom+(Top−Bottom)/(1+10^{logEC50-X}) where X is log(compound concentration), Y is % inhibition, and Bottom and Top are the lower and upper plateaux. Kₘ concentrations were also determined by non-linear regression.

### Example 7

### DYRK2 Inhibitor Assay

Test compounds in 2.5 μl (0.001-67 μM final concentration) were incubated in 25 μl in the presence of 50 mM Tris-HCl, pH 7.5, 5 mM MgCl₂, 10 μM ATP (K_{M} value), trace amounts of radioactively labeled γ³³P-ATP (50 nM, PerkinElmer), 10 nM GST-DYRK2 enzyme (Carna Biosciences, Japan) and 150 μM biotin-Woodtide peptide substrate (biotin-KKISGRLSPIMTEQ-NH2, Abgent, at K_{M} value) at room temperature. Reactions were stopped after 10 minutes by addition of 30 mM EDTA followed by spotting 10 μl of the reaction mix on to P81 phosphocellulose filter (Whatman). P81 filters were washed three times for 10 min in 0.75% phosphoric acid to remove free γ³³P-ATP and then air-dried. γ³³P-ATP incorporation was measured using a MicroBeta liquid scintillation counter (PerkinElmer). Background level of ³³P incorporation was defined from control reaction lacking peptide.

### Example 8

### IC₅₀ Determination for Lead Compound LDN-192960

In order to study the role of haspin's kinase activity in mitosis (and other cellular processes) and its potential role in cancer, identification and optimization of inhibitors was first necessary. Utilizing time-resolved fluorescence resonance energy transfer (TR-FRET) high throughput screening (HTS) assay with histone H3 peptide as substrate and a europium-labeled phosphospecific monoclonal antibody for detecting phosphorylated substrate (H3T3ph), the acridine derivative LDN-192960 was discovered as a potent inhibitor (IC₅₀=0.010 μM). Kinase profiling of LDN-192960 revealed potent DYRK2 inhibitory activity as well.

### Example 9

### IC₅₀ Determination of Compound LDN-192960 Against Profile Panel Kinases

Compound LDN-192960 was initially profiled for functional inhibitory activity against a panel of two hundred and seventy kinases at 10 μM. The results demonstrated that this compound was selective and only inhibited ten of the other kinases by ≧90%. Also, an interaction map for compound LDN-192960 was produced. A kinase dendrogram was generated using percent inhibition values versus controls and the ‘TreeSpot’ kinome data visualization tool available as a web-based application (see the worldwide webpage kinomescan.com/login.aspx). Only kinases with percent control values <30% were displayed. Although haspin was not available in the original profile, it was subsequently found to give 100% inhibition of haspin activity in the Carna Bioscience assay at 10 μM. The kinase dendrogram was adapted by KINOMEscan and is reproduced with permission from Science (see the worldwide webpage sciencemag.org) and Cell Signaling Technology, Inc. (see the worldwide webpage cellsignal.com).

IC₅₀ values were determined for these kinases (Table 1 below), with only five being potently inhibited (IC₅₀<1 μM). DYRK2 was the most sensitive of these kinases (IC₅₀=2 nM).

TABLE 1

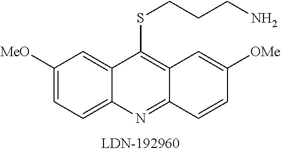 [SMILES: COc1ccc2nc3ccc(OC)cc3c(SCCCN)c2c1]

| Kinase | IC₅₀ (μM) | Kinase | IC₅₀ (μM) |
|---|---|---|---|
| TRKB | 91 | ROS | 1.6 |
| CLK1 | 0.21 | HIPK1 | 1.4 |
| DYRK1A | 0.10 | HIPK2 | 1.3 |
| DYRK2 | 0.002 | PIM1 | 0.72 |
| DYRK3 | 0.019 | PIM2 | 56 |

### Example 10

### Synthesis of Analogs of LDN-192960

Acridine analogs were prepared according to the methods outlined in schemes 1-4 below. The synthesis of many of the acridine analogs was accomplished according to Scheme 1. 2-Bromobenzoic acids 2 were coupled to anilines 3 using a copper-mediated procedure to give 4. Cyclization of 4 to 9-chloroacridines 5 was accomplished using phosphorus oxychloride. Treatment of 5 with P₄S₁₀ in the presence of DMPU gave 6. Alternatively, acid 4 was cyclized to ketone 7 in the presence of polyphosphoric acid (PPA), which was subsequently treated with Lawesson's reagent with microwave (MW) heating at 110° C. to produce 6. The thioketone 6 could be alkylated with various amino-protected alkylbromides (BrCH₂(CH2)ₙY; Y=NHBoc, NMeBoc, or NPhthalimide) in the presence of base (KOH) and the phase transfer catalyst tetrabutylammonium iodide (TBAI) in a mixture of toluene and water to give 8. Boc-protected analogs of 8 (Y=NHBoc or NMeBoc) upon treatment of 4N HCl in a mixture of 1,4-dioxane and methanol gave 9 (Y=NH₂ or NHMe). Alternatively for analogs of 9 with Y=NH₂, they could also be prepared directly from 6 via alkylation.

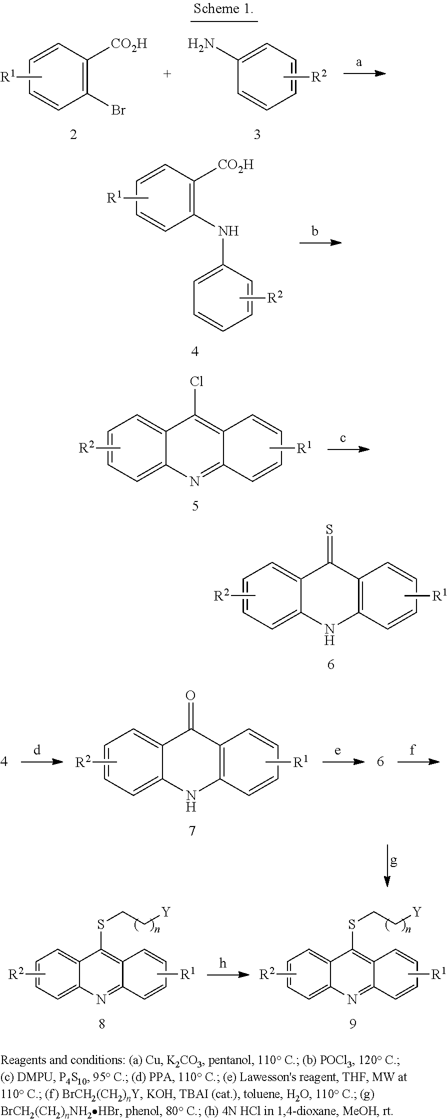

Acidine analogs where the alkylamine groups were connected through an O or NH were prepared according to Scheme 2. Ketone 10 was converted to 11 as previously described. Then a nucleophilic aromatic substitution with a mono-protected diamine followed by removal of the protecting group gave 12. Ketone 10 was also alkylated with N-Boc-protected 1-amino-3-bromopropane in the presence of base (KOH) and TBAI followed by de-protection to give 13.

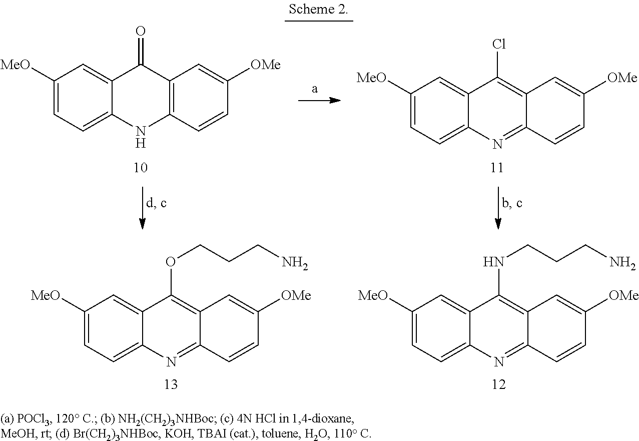 [SMILES: COc1ccc2[nH]c3ccc(OC)cc3c(=O)c2c1 ; COc1ccc2nc3ccc(OC)cc3c(Cl)c2c1 ; COc1ccc2nc3ccc(OC)cc3c(OCCCN)c2c1 ; COc1ccc2nc3ccc(OC)cc3c(NCCCN)c2c1]

The synthesis of acridine analogs where the alkylamine group was connected through a methylene was prepared according to Scheme 3. Diphenylamine derivative 14 was condensed with acetic acid to give acridine analog 15. The methyl substituent in the 9-position was oxidized with selenium dioxide to give aldehyde 16. Addition of the anion of N-Boc protected alkyne gave alcohol 17. Exposure of 17 to reducing conditions (Pd/C and Et₃SiH) resulted in reduction of the alkyne and alcohol. Finally removal of the protecting group on the amine yielded 18.

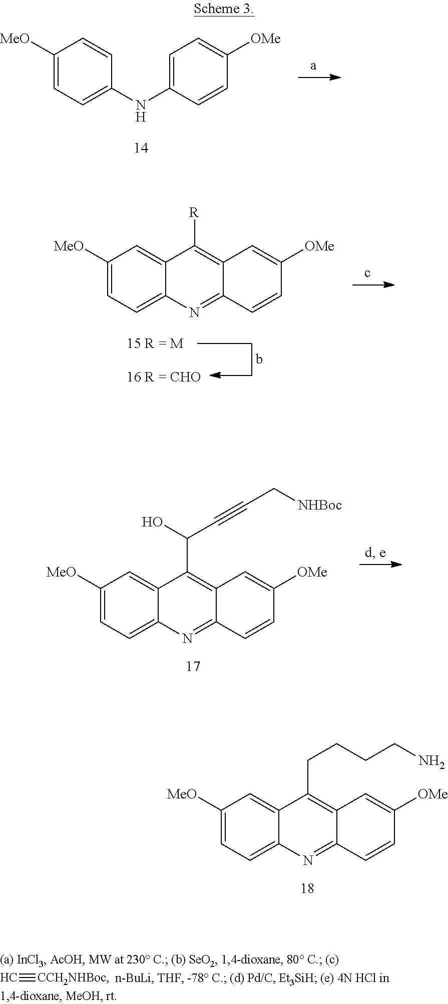

The synthesis of a tetrahydroacridine analog is outlined in Scheme 4. β-Ketoester 19 was allowed to react with 4-anisidine, 20, to produce 21. Cyclization of 21 in sulfuric acid gave ketone 22. This material was converted to the corresponding thioketone 23. Alkylation with 1-amino-3-bromopropane hydrobromide gave 24.

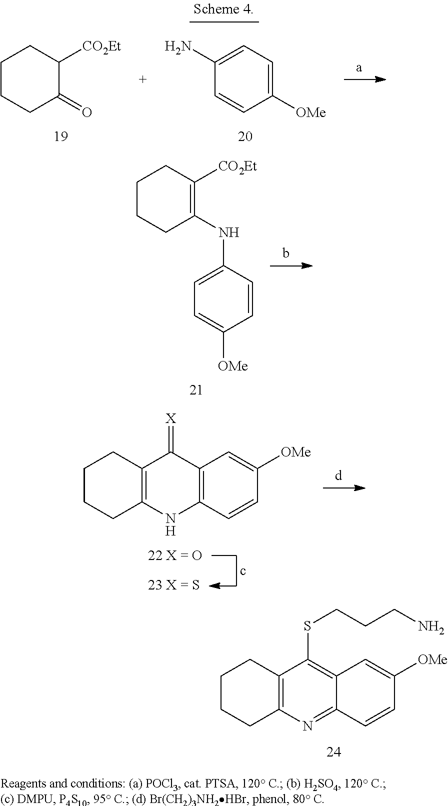

Finally, a 2-phenylquinoline analog was synthesized according to Scheme 5. The acetophenone derivative 25 was coupled with benzamide in the presence of a catalytic amount of CuI to give 26. A base-mediated cyclization of 26 gave 27. Conversion of this material to the thiol analog 28 was accomplished with Lawesson's reagent. Then alkylation and deprotection as previously described for other analogs yielded 29.

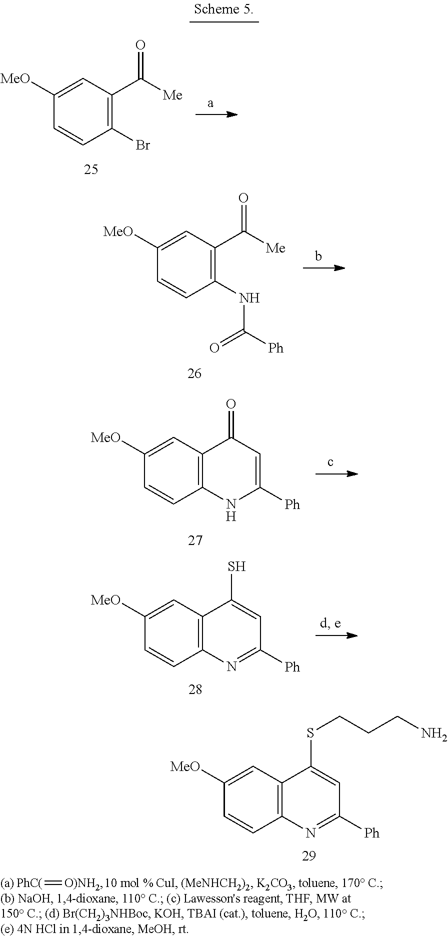

### Example 11

### Characterization of Acridine Analogs

The acridine analogs prepared in Example 10 were characterized by ¹H-NMR.

| LDN number | Structure |
|---|---|
| LDN-192960 | 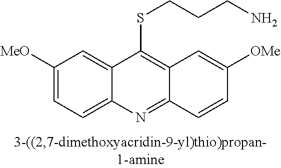 [SMILES: COc1ccc2nc3ccc(OC)cc3c(SCCCN)c2c1] |

¹H NMR (300 MHz, DMSO-d₆) δ ppm: 1.65 (t, 2H, —CH2—), 2.81 (sx, 2H, —CH2—
NH2), 3.11 (t, 2H, —S—CH2—), 4.04 (s, 6H, OCH3), 5.50 (bs, 2H, NH2), 7.72-7.77
(dd, 2H, J = 2.53, 9.35 Hz, C—H_{3,6}), 7.85 (d, 2H, J = 2.65 Hz, C—H_{1,8}), 8.21-8.26 (d,
2H, J = 9.35 Hz, C—H_{4,5}).

| | |
|---|---|
| LDN-209856 | 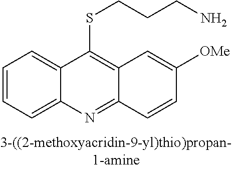 [SMILES: COc1ccc2nc3ccccc3c(SCCCN)c2c1] |

¹H NMR (300 MHz, DMSO-d₆) δ ppm: 1.71 (t, 2H, —CH2—), 2.79 (sx, 2H, —CH2—
NH2), 3.25 (t, 2H, —S—CH2—), 4.07 (s, 6H, OCH3), 7.70 (bs, 2H, NH3+), 7.83-7.90 (dd,
1H, J = 2.65, 9.35 Hz, C—H₃), 7.09 (dt, 1H, C—H₇), 7.96 (d, 1H, J = 2.65 Hz, C—H₁), 8.30-
8.35 (d, 1H, J = 9.35 Hz, C—H₄), 8.32-8.37 (d, 1H, J = 8.34 Hz, C—H₅), 8.77-8.81 (d, 1H,
J = 8.09 Hz, C—H₈).

| | |
|---|---|
| LDN-192965 | 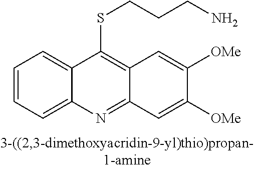 [SMILES: COc1cc2nc3ccccc3c(SCCCN)c2cc1OC] |

¹H NMR (300 MHz, DMSO-d₆) δ ppm: 1.66 (t, 2H, —CH2—), 2.77 (sx, 2H, —CH2—
NH2), 3.24 (t, 2H, —S—CH2—), 4.08 (s, 3H, OCH3), 4.11 (s, 3H, OCH3), 7.62 (s, 1H,
C—H₄), 7.73 (bs, 3H, NH3+), 7.79 (s, 1H, C—H₁), 7.87 (dt, 1H, C—H₇), 8.12 (dt, 1H,
C—H₆), 8.28 (dd, 1H, C—H₅), 8.68 (dd, 1H, C—H₈).

| | |
|---|---|
| LDN-209838 | 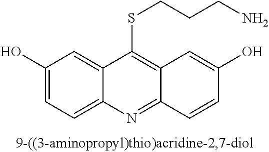 [SMILES: NCCCSc1c2cc(O)ccc2nc2ccc(O)cc12] |

¹H NMR (300 MHz, DMSO-d₆) δ ppm: 1.66 (t, 2H, —CH2—), 2.81 (sx, 2H, —CH2—
NH2), 3.16 (t, 2H, —S—CH2—), 7.65 (bs, 2H, NH2), 7.68-7.74 (dd, 2H, J = 2.53, 9.35 Hz,
C—H_{3,6}), 7.95 (d, 2H, J = 2.65 Hz, C—H_{1,8}), 8.20-8.25 (d, 2H, J = 9.35 Hz, C—H_{4,5}), 10.94
(bs, 2H, OH).

| | |
|---|---|
| LDN-209839 | 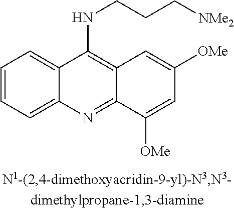 [SMILES: COc1cc(OC)c2nc3ccccc3c(NCCCN(C)C)c2c1] |

¹H NMR (300 MHz, DMSO-d₆) δ ppm: 2.40 (m, 2H, —CH2—), 2.71 (s, 6H, N—CH3),
3.24 (m, 2H, —CH2—), 4.05 (s, 3H, OCH3), 4.14 (s, 3H, OCH3), 4.21 (m, 2H, —CH2—),
7.19 (s, 1H, C—H₃), 7.52 (s, 1H, C—H₁), 7.79 (t, 1H, C—H₇), 7.89 (t, 1H, C—H₆), 8.32
(d, 1H, C—H₃), 8.46 (d, 1H, C—H₈), 10.01 (m, 1H, NH).

| | |
|---|---|
| LDN-209840 | 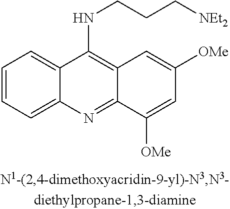 |

¹H NMR (300 MHz, DMSO-d₆) δ ppm: 1.21 (t, 6H, CH3), 2.40 (m, 2H, —CH2—),
2.82 (q, 4H, N—CH2), 3.24 (m, 2H, —CH2—), 4.04 (s, 3H, OCH3), 4.14 (s, 3H, OCH3),
4.23 (m, 2H, —CH2—), 7.20 (s, 1H, C—H₃), 7.52 (s, 1H, C—H₁), 7.79 (m, 2H, C—H_{6,7}),
8.30 (d, 1H, C—H₅), 8.50 (d, 1H, C—H₈), 9.03 (m, 1H, NH).

| | |
|---|---|
| LDN-209928 | 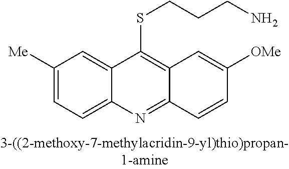 [SMILES: COc1ccc2nc3ccc(C)cc3c(SCCCN)c2c1] |

¹H NMR (300 MHz, DMSO-d₆) δ ppm: 1.66 (t, 2H, —CH2—), 2.64 (s, 3H, CH3),
2.80 (sx, 2H, —CH2—NH2), 3.21 (t, 2H, —S—CH2—), 4.06 (s, 3H, OCH3), 7.67 (bs, 2H,
NH2), 7.78-7.82 (dd, 1H, C—H₆), 7.90-7.96 (dd, 1H, C—H₃),7.93 (d, 1H, C—H₈),
8.21-8.24 (d, 1H, C—H₅), 8.25-8.28 (d, 1H, C—H₄), 8.51 (s, 1H, C—H₁).

| | |
|---|---|
| LDN-209929 | 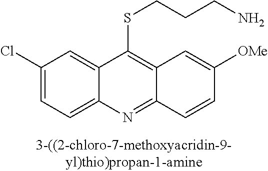 [SMILES: COc1ccc2nc3ccc(Cl)cc3c(SCCCN)c2c1] |

¹H NMR (300 MHz, DMSO-d₆) δ ppm: 1.60-1.70 (q, 2H, J = 7.55 Hz, CH₂ Beta),
2.75-2.81 (q, 2H, CH₂ Gamma), 3.09 (t, 2H, J = 7.56 Hz, CH₂ Alpha), 4.04 (s, 3H, O—CH₃),
7.64-7.68 (dd, 1H, J = 2.83, 9.44 Hz, C—H₆), 7.85-7.89 (dd, 1H, J = 2.46, 9.26 Hz,
C—H₃), 7.89 (d, 1H, J = 2.44 Hz, C—H₈), 8.14-8.17 (d, 1H, J = 9.44 Hz, C—H₅), 8.21-8.24
(d, 1H, J = 9.26 Hz, C—H₄), 8.58 (d, 1H, J = 2.27 Hz, C—H₁).

| | |
|---|---|
| LDN-211840 | 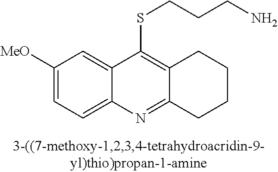 [SMILES: COc1ccc2nc3c(c(SCCCN)c2c1)CCCC3] |

¹H NMR (300 MHz, DMSO-d₆) δ ppm: 1.74 (t, 2H, —CH2—), 1.91 (m, 4H, —CH2—),
2.84 (sx, 2H, —CH2—NH2), 3.13-3.24 (m, 6H, —S—CH2—, —CH2—), 4.00 (s, 3H,
OCH3), 7.65-7.71 (dd, 1H, J = 2.65, 9.35 Hz, C—H₃), 7.79 (d, 1H, J = 2.65 Hz, C—H₁),
8.12-8.17 (d, 1H, J = 9.35 Hz, C—H₄).

| | |
|---|---|
| LDN-211848 | 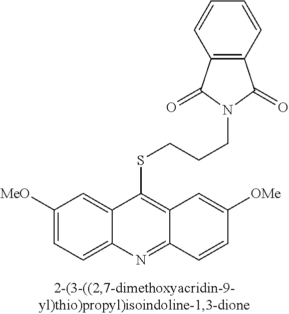 [SMILES: COc1ccc2nc3ccc(OC)cc3c(SCCCN3C(=O)c4ccccc4C3=O)c2c1] |

¹H NMR (300 MHz, DMSO-d₆) δ ppm: 1.71 (sx, 2H, —CH2—), 3.09 (s, 2H, S—CH2—),
3.61 (t, 2H, N—CH2—), 3.99 (s, 6H, OCH3), 7.46-7.52 (dd, 2H, J = 2.53, 9.35 Hz, C—H_{3,6}),
7.78 (m, 4H, Chphtal), 7.87 (d, 2H, J = 2.65 Hz, C—H_{1,8}), 8.03-8.07 (d, 2H, J = 9.35 Hz,
C—H_{4,5}).

| | |
|---|---|
| LDN-211849 | 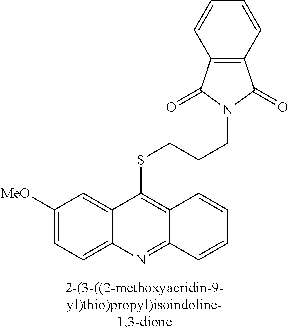 [SMILES: COc1ccc2nc3ccccc3c(SCCCN3C(=O)c4ccccc4C3=O)c2c1] |

¹H NMR (300 MHz, DMSO-d₆) δ ppm: 1.70 (sx, 2H, —CH2—), 3.05 (s, 2H, S—CH2—),
3.60 (t, 2H, N—CH2—), 3.99 (s, 6H, OCH3), 7.49-7.55 (dd, 1H, J = 2.53, 9.35 Hz, C—H₃),
7.71 (dt, 1H, C—H₇), 7.75 (dt, 1H, C—H₆), 7.79 (m, 4H, C-Hnaphtyl), 7.91 (d, 1H, J = 2.65
Hz, C—H₁), 8.05-8.09 (d, 1H, J = 9.35 Hz, C—H₄), 8.11-8.15 (d, 1H, J = 8.35 Hz, C—H₅),
8.64-8.68 (d, 1H, J = 8.35 Hz, C—H₈).

| | |
|---|---|
| LDN-212055 | 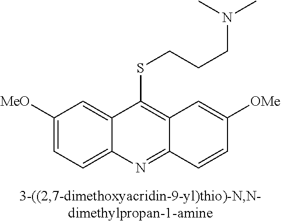 [SMILES: COc1ccc2nc3ccc(OC)cc3c(SCCCN(C)C)c2c1] |

¹H NMR (300 MHz, DMSO-d₆) δ ppm: 1.85 (sx, 2H, —CH2—), 2.59 (s, 6H, N—CH3),
3.01 (s, 2H, S—CH2—), 3.19 (t, 2H, N—CH2—), 4.06 (s, 6H, OCH3), 7.70-7.76 (dd,
2H, J = 2.53, 9.35 Hz, C—H_{3,6}), 7.91 (d, 2H, J = 2.65 Hz, C—H_{1,8}), 8.32-8.36 (d, 2H,
J = 9.35 Hz, C—H_{4,5}).

### Example 12

### IC₅₀ Determination for Haspin and DYRK2 Kinase Inhibition

IC₅₀ values were determined for inhibition of Haspin and DYRK2 kinases utilizing the TR-FRET assay described in Example 3 and the DYRK2 assay described in Example 7. The results are reported in Table 2.

TABLE 2

| | | Haspin | DYRK2 |
|---|---|---|---|
| LDN number | Structure | IC₅₀ (μM) | IC₅₀ (μM) |
| LDN-192960 | 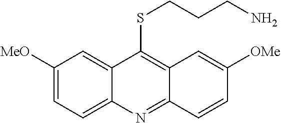 [SMILES: COc1ccc2nc3ccc(OC)cc3c(SCCCN)c2c1] | <0.050 | <0.050 |
| LDN-209856 | 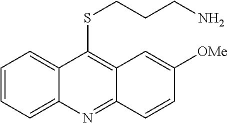 [SMILES: COc1ccc2nc3ccccc3c(SCCCN)c2c1] | <0.050 | <0.20 |
| LDN-192965 | 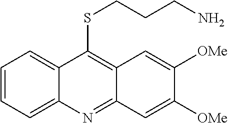 [SMILES: COc1cc2nc3ccccc3c(SCCCN)c2cc1OC] | >20 | >10 |
| LDN-209838 | 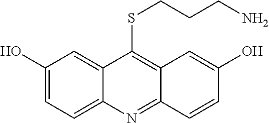 [SMILES: NCCCSc1c2cc(O)ccc2nc2ccc(O)cc12] | <0.050 | <0.20 |
| LDN-209839 | 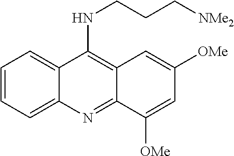 [SMILES: COc1cc(OC)c2nc3ccccc3c(NCCCN(C)C)c2c1] | >20 | >10 |
| LDN-209840 | 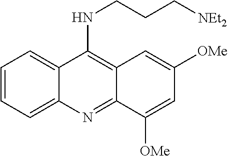 | >20 | >1.0 |
| LDN-209928 | 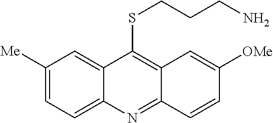 [SMILES: COc1ccc2nc3ccc(C)cc3c(SCCCN)c2c1] | <0.100 | <0.500 |
| LDN-209929 | 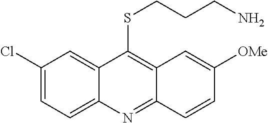 [SMILES: COc1ccc2nc3ccc(Cl)cc3c(SCCCN)c2c1] | <0.100 | <10 |
| LDN-209957 | 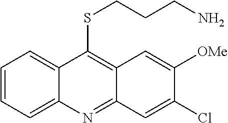 [SMILES: COc1cc2c(SCCCN)c3ccccc3nc2cc1Cl] | <0.100 | <10 |
| LDN-209958 | 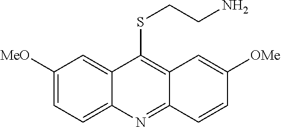 [SMILES: COc1ccc2nc3ccc(OC)cc3c(SCCN)c2c1] | <0.20 | <0.50 |
| LDN-209959 | 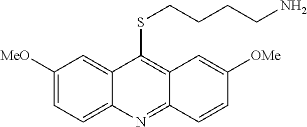 [SMILES: COc1ccc2nc3ccc(OC)cc3c(SCCCCN)c2c1] | <0.050 | <0.050 |
| LDN-209960 | 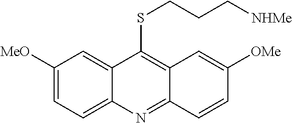 [SMILES: CNCCCSc1c2cc(OC)ccc2nc2ccc(OC)cc12] | <0.010 | <0.200 |
| LDN-209961 | 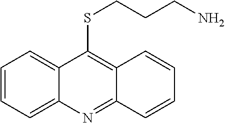 [SMILES: NCCCSc1c2ccccc2nc2ccccc12] | <10 | <10 |
| LDN-209962 | 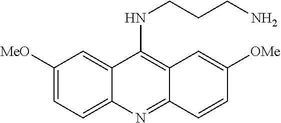 [SMILES: COc1ccc2nc3ccc(OC)cc3c(NCCCN)c2c1] | <5 | <10 |
| LDN-209963 | 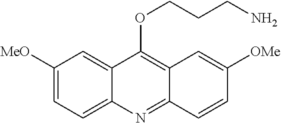 [SMILES: COc1ccc2nc3ccc(OC)cc3c(OCCCN)c2c1] | >20 | >20 |
| LDN-209964 | 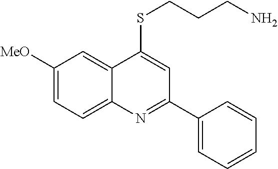 [SMILES: COc1ccc2nc(-c3ccccc3)cc(SCCCN)c2c1] | <10 | >10 |
| LDN-209973 | 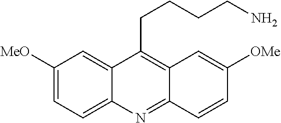 [SMILES: COc1ccc2nc3ccc(OC)cc3c(CCCCN)c2c1] | <0.050 | <0.50 |
| LDN-211840 | 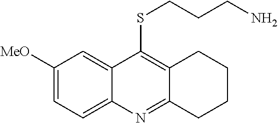 [SMILES: COc1ccc2nc3c(c(SCCCN)c2c1)CCCC3] | <0.50 | <10 |
| LDN-211848 | 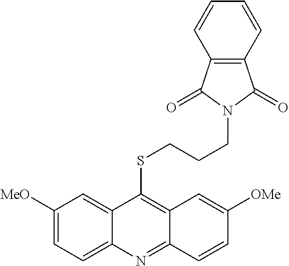 [SMILES: COc1ccc2nc3ccc(OC)cc3c(SCCCN3C(=O)c4ccccc4C3=O)c2c1] | <10 | <0.50 |
| LDN-211849 | 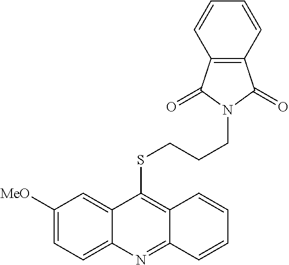 [SMILES: COc1ccc2nc3ccccc3c(SCCCN3C(=O)c4ccccc4C3=O)c2c1] | <10 | <1.0 |
| LDN-212055 | 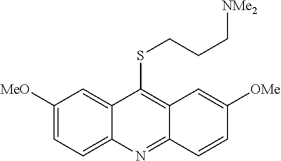 [SMILES: COc1ccc2nc3ccc(OC)cc3c(SCCCN(C)C)c2c1] | <0.050 | <0.50 |

### Example 13

### Structure-Activity Relationship Study

The human haspin kinase inhibitory activity of the various compounds was evaluated using the same assay utilized for the HTS, except in the presence of varying test compound concentrations. DYRK2 kinase inhibitory activity was measured by ³³P-incorporation into Woodtide peptide substrate in the presence of human DYRK2 containing an N-terminal GST-fusion protein and γ³³P-ATP.

Only one of the methoxy groups in compound LDN-192960 was necessary for potent haspin inhibition. When both of the methoxy groups were removed as in compound LDN-209961 inhibitory activity was dramatically reduced (Table 2, above). However, when only one of the methoxy groups was removed (compound LDN-209856) or replaced with a methyl (compound LDN-209928) or chlorine (compound LDN-209929) potent activity (IC₅₀<100 nM) was retained. The methoxy substituents of compound LDN-192960 could also be replaced with hydroxyl groups (compound LDN-209838).

Transposition of the 7-methoxy to the 3-position (compound LDN-192965) resulted in loss of activity. However, the 2-methoxy-3-chloro analog (compound LDN-209957) was still quite active. The three aromatic rings that comprise the acridine also appeared necessary. Both compounds 24 and 29 (Schemes 4 and 5) lacked haspin inhibitory activity. Next, the tether length between the thioether at the 9-position of the acridine and the primary amine was examined. Truncation (compound LDN-209958) resulted in reduced activity, while addition of another methylene unit (compound LDN-209959) had only a minimal impact on potency. The contribution of the amine was also examined. A secondary amine (compound LDN-209960) was equally potent and a tertiary amine (compound 212055) only resulted in a slight decrease in activity. However, incorporation of the nitrogen into a phthalimide (compounds LDN-211848 and LDN-211849) resulted in a significant loss of activity.

Additionally, the thioether was examined. Replacement of the thioether with an amine (compound LDN-209962) or ether (compound LDN-209963) was detrimental. However, replacement of the sulfur with a methylene (compound LDN-209973) retained potent inhibitory activity. The SAR for DYRK2 inhibition had many similarities to that observed for haspin inhibition with some notable exceptions. Both methoxy groups appear to be necessary for DYRK2 inhibitory activity. For example, removal of both methoxy groups (compound LDN-209961) was very detrimental, while removal of one methoxy (compound LDN-209856) still resulted in a significant erosion of potency. Likewise, replacement of one methoxy with a methyl (compound LDN-209928) or a chlorine atom (compound LDN-209929) was not as tolerated as compared to the results observed for haspin inhibition. Transposition of the 7-methoxy to the 3-position (compound LDN-192965) also lead to loss in activity. Similarly, and unlike in the haspin SAR, removal of the 7-methoxy and addition of a 3-chloro group (compound LDN-209957) was not tolerated for DYRK2 inhibition. The three aromatic rings that comprise the acridine also appear necessary for DYRK2 inhibition, with both compounds 24 and 29 lacking activity. The effect of the tether length between the thioether at the 9-position of the acridine and the primary amine (compounds LDN-209958 and LDN-209959) was the same as previously observed for haspin inhibition. For DYRK2 inhibition the primary amine was better than the secondary amine (compound LDN-209960) or tertiary (compound LDN-212055) amines. Incorporation of the nitrogen into a phthalimide resulted in a less dramatic impact on DYRK2 inhibition compared to haspin and provided a moderately potent analog (compound LDN-211848) that was 5.4-fold selective for DYRK2 versus haspin. Similar to the observations made with haspin inhibition, replacement of the thioether with an amine (compound LDN-209962) or ether (compound LDN-209963) was detrimental to DYRK2 inhibition, while replacement of the sulfur with a methylene (LDN-209973) was tolerated, albeit with a 5-fold reduction in potency.

This SAR study revealed that several structural features of LDN-192960, such as the three acridine aromatic rings, the presence of one or both methoxy groups, a three or four methylene tether between the thioether and the acridine, and a thioether or CH₂ (but not an amine or ether) link to the acridine were necessary for both haspin and DYRK2 inhibition. However, several structural differences were noted that allowed generation of a potent haspin kinase inhibitor (compound LDN-209929, IC₅₀<60 nM) with 180-fold selectivity versus DYRK2. In addition, a moderately potent DYRK2 inhibitor (compound 211848, IC₅₀<400 nM) with a 5.4-fold selectivity versus haspin was also identified.

### Example 14

### Data Obtained From the National Cancer Institute

The screening is a two-stage process, beginning with the evaluation of all compounds against the 60 cell lines at a single dose of 10 uM. The output from the single dose screen is reported as a mean graph and is available for analysis by the COMPARE program. Compounds which exhibit significant growth inhibition are evaluated against the 60 cell panel at five concentration levels.

### Methodology of the In Vitro Cancer Screen

The human tumor cell lines of the cancer screening panel were grown in RPMI 1640 medium containing 5% fetal bovine serum and 2 mM L-glutamine. For a typical screening experiment, cells were inoculated into 96 well microtiter plates in 100 μL at plating densities ranging from 5,000 to 40,000 cells/well depending on the doubling time of individual cell lines. After cell inoculation, the microtiter plates were incubated at 37° C., 5% CO2, 95% air and 100% relative humidity for 24 hours prior to addition of compounds.

After 24 hours, two plates of each cell line were fixed in situ with TCA, to represent a measurement of the cell population for each cell line at the time of compound addition (Tz). The compounds were solubilized in dimethyl sulfoxide at 400-fold the desired final maximum test concentration and stored frozen prior to use. At the time of compound addition, an aliquot of frozen concentrate was thawed and diluted to twice the desired final maximum test concentration with complete medium containing 50 μg/ml gentamicin. Additional four, 10-fold or half-log serial dilutions were made to provide a total of five compound concentrations plus control. Aliquots of 100 μl of these different compound dilutions were added to the appropriate microtiter wells already containing 100 μl of medium, resulting in the required final compound concentrations.

Following compound addition, the plates were incubated for an additional 48 hours at 37° C., 5% CO2, 95% air, and 100% relative humidity. For adherent cells, the assay is terminated by the addition of cold TCA. Cells were fixed in situ by the gentle addition of 50 μl of cold 50% (w/v) TCA (final concentration, 10% TCA) and incubated for 60 minutes at 4° C. The supernatant was discarded, and the plates were washed five times with tap water and air dried. Sulforhodamine B (SRB) solution (100 μl) at 0.4% (w/v) in 1% acetic acid was added to each well, and plates were incubated for 10 minutes at room temperature. After staining, unbound dye was removed by washing five times with 1% acetic acid and the plates were air dried. Bound stain was subsequently solubilized with 10 mM trizma base, and the absorbance was read on an automated plate reader at a wavelength of 515 nm. For suspension cells, the methodology was the same except that the assay was terminated by fixing settled cells at the bottom of the wells by gently adding 50 μl of 80% TCA (final concentration, 16% TCA). Using the seven absorbance measurements [time zero, (Tz), control growth, (C), and test growth in the presence of compound at the five concentration levels (Ti)], the percentage growth was calculated at each of the compound concentrations levels. Percentage growth inhibition was calculated as:

[(Ti−Tz)/(C−Tz)]×100 for concentrations for which Ti>/=Tz

[(Ti−Tz)/Tz]×100 for concentrations for which Ti<Tz.

Three dose response parameters were calculated for each experimental agent. Growth inhibition of 50% (GI50) was calculated from [(Ti−Tz)/(C−Tz)]×100=50, which was the compound concentration resulting in a 50% reduction in the net protein increase (as measured by SRB staining) in control cells during the compound incubation. The compound concentration resulting in total growth inhibition (TGI) was calculated from Ti=Tz. The LC₅₀ (concentration of compound resulting in a 50% reduction in the measured protein at the end of the compound treatment as compared to that at the beginning) indicating a net loss of cells following treatment was calculated from [(Ti−Tz)/Tz]×100=−50. Values were calculated for each of these three parameters if the level of activity was reached; however, if the effect was not reached or was exceeded, the value for that parameter was expressed as greater or less than the maximum or minimum concentration tested.

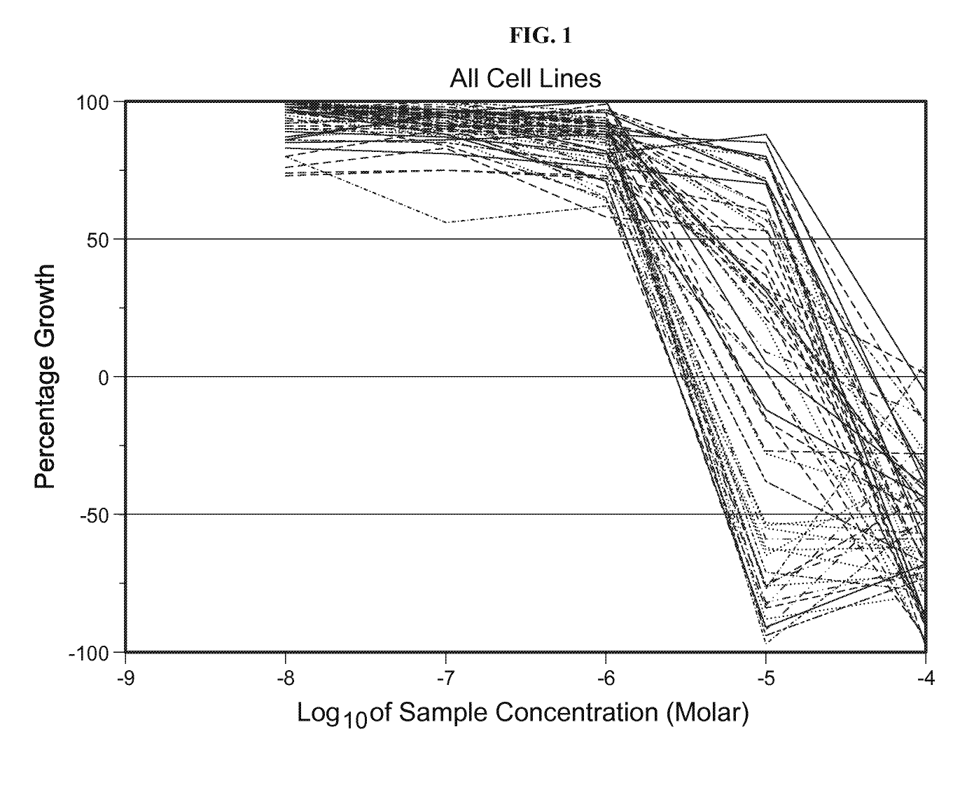

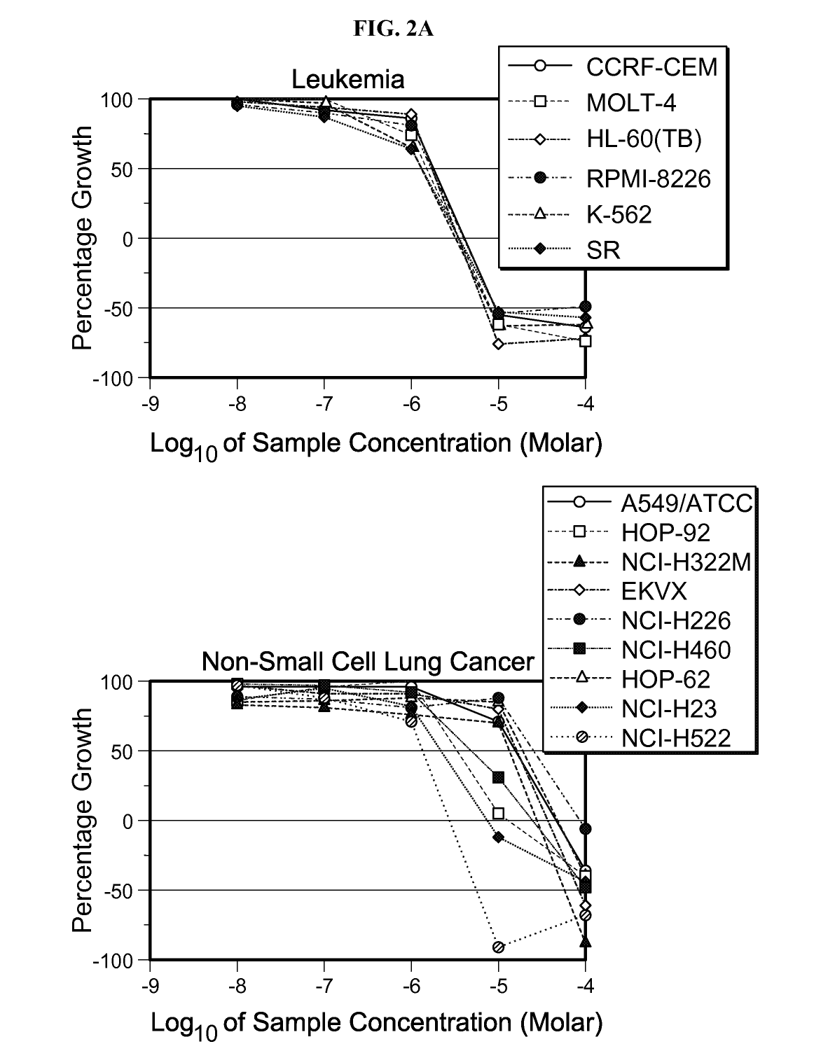

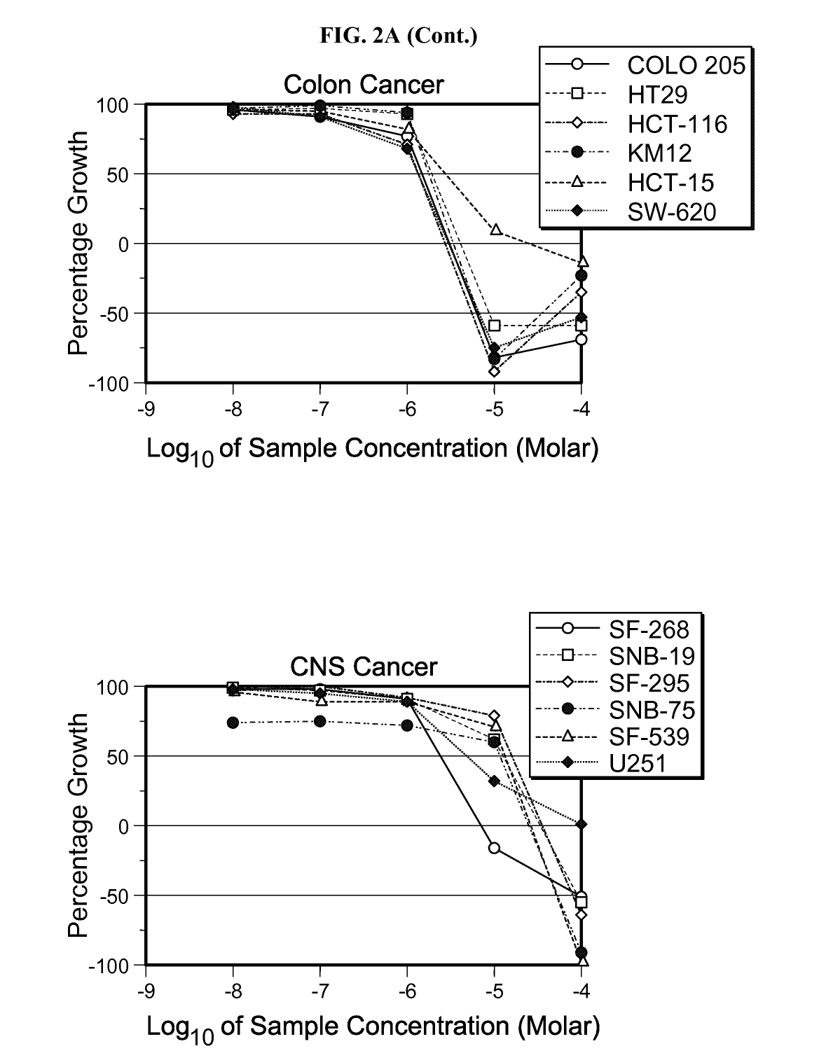

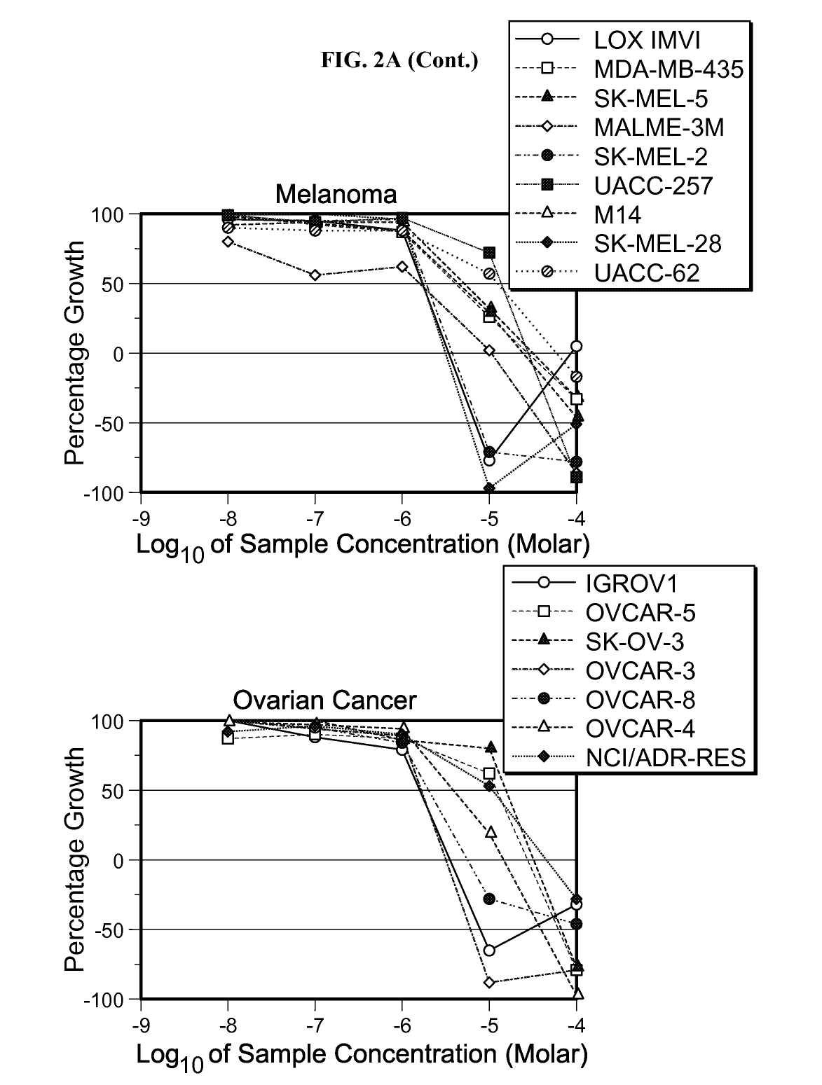

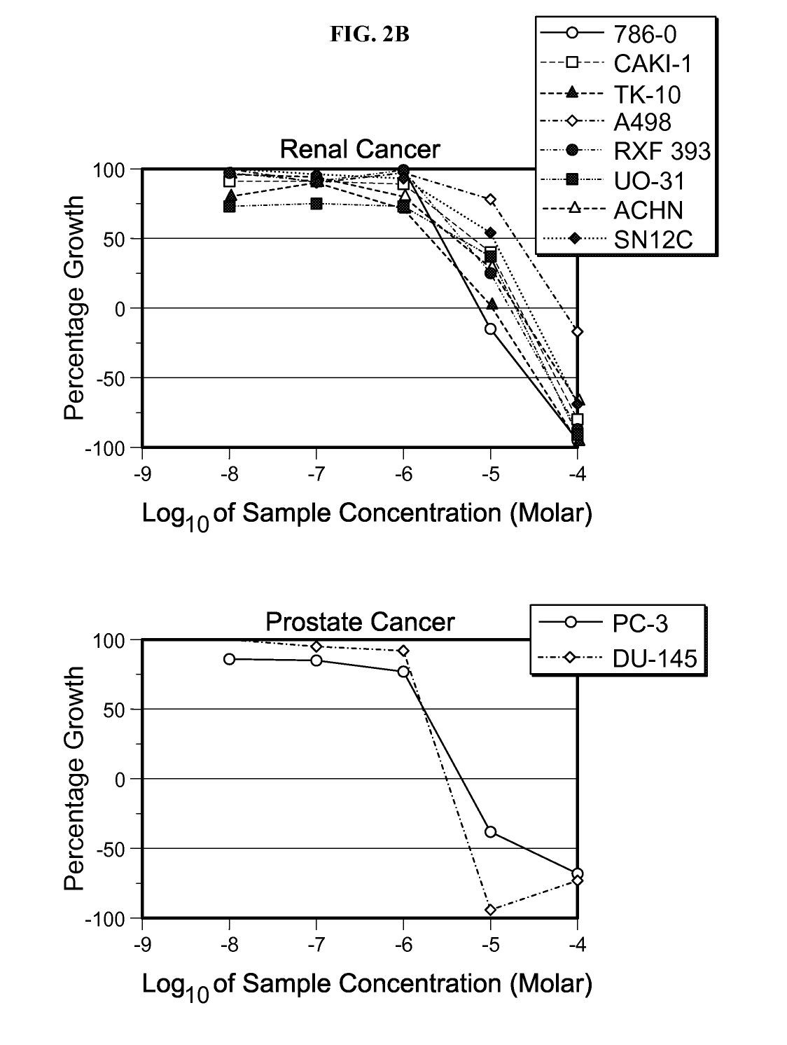

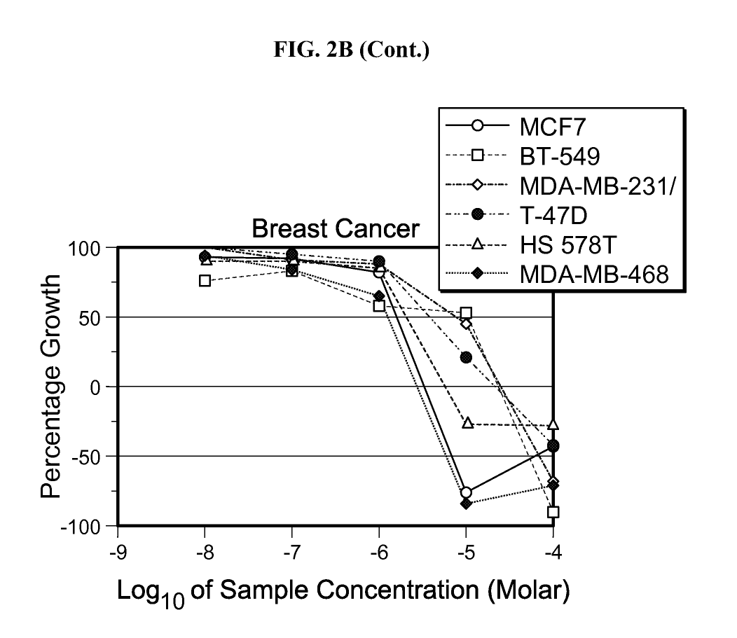

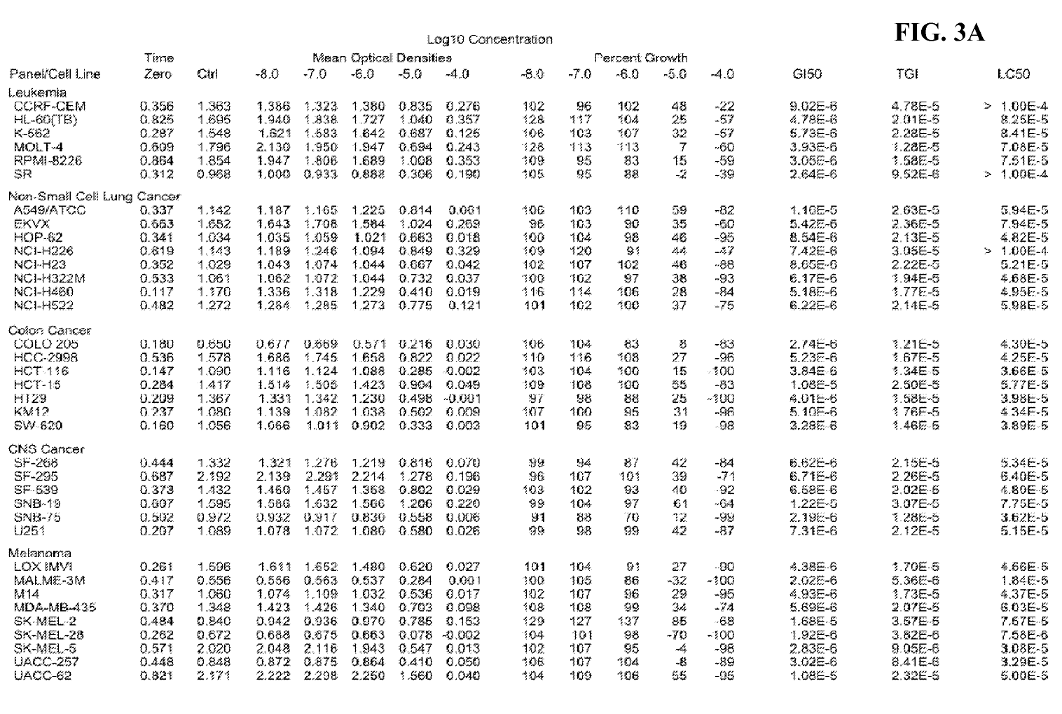

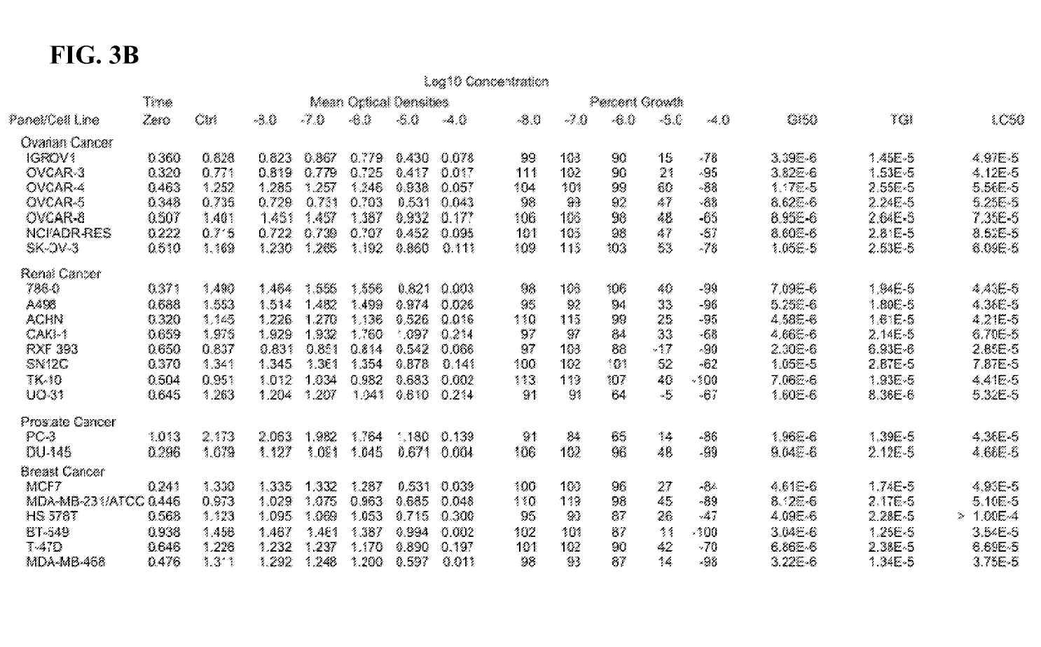

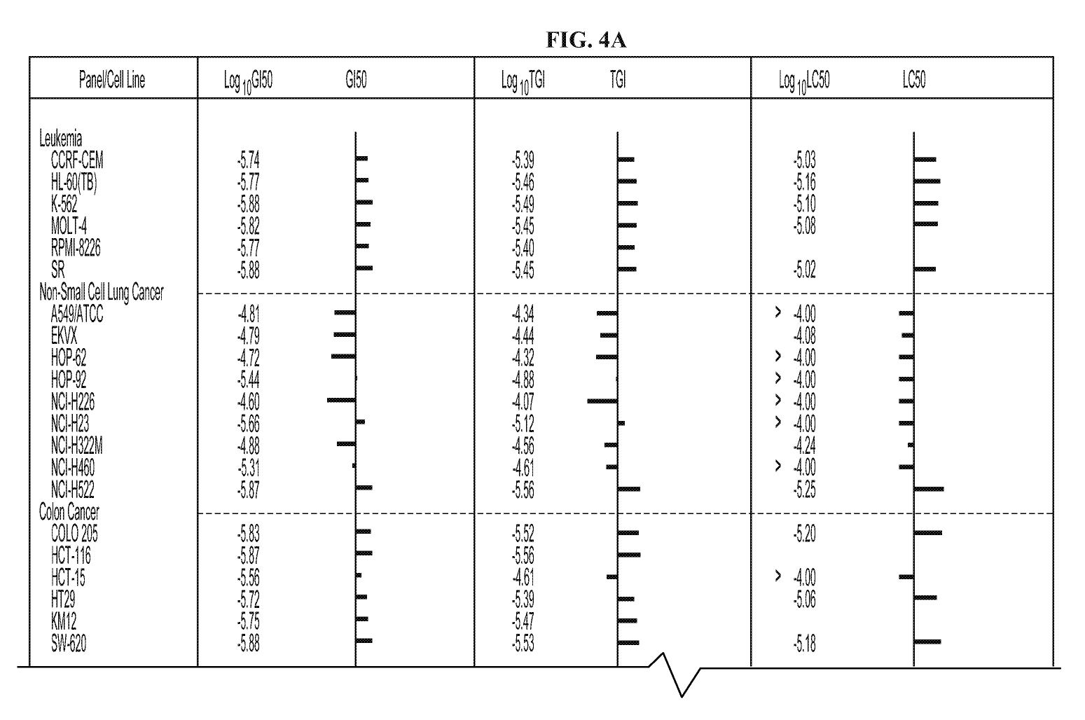

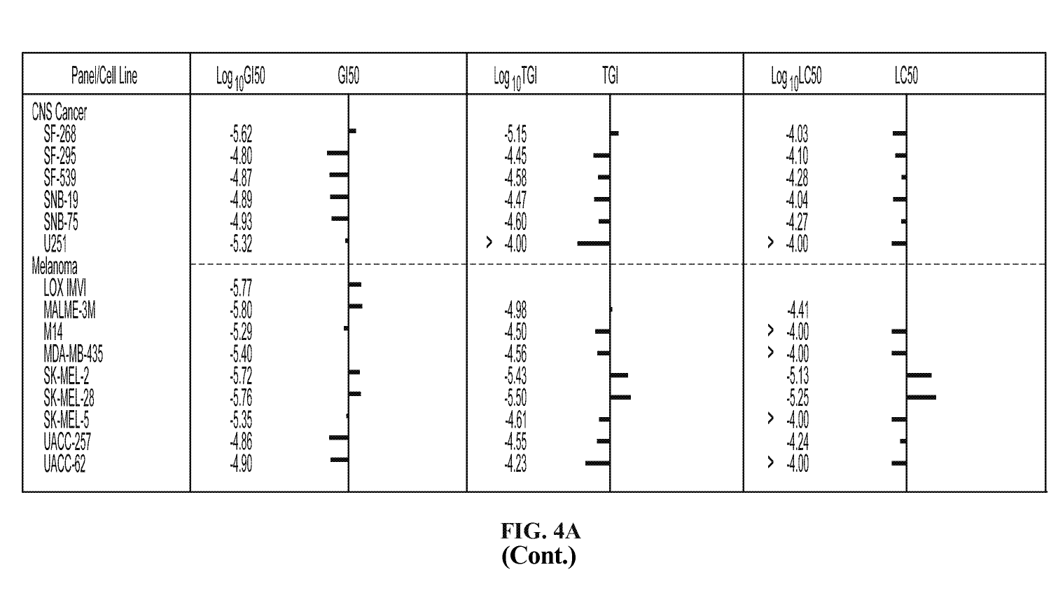

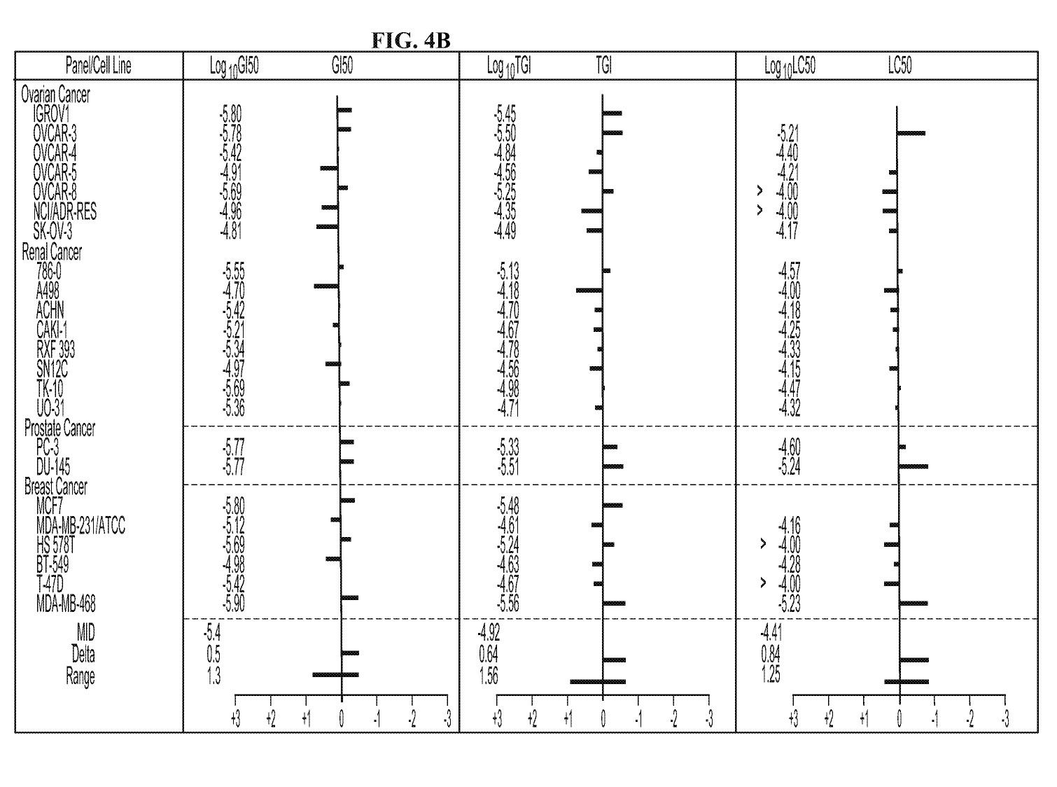

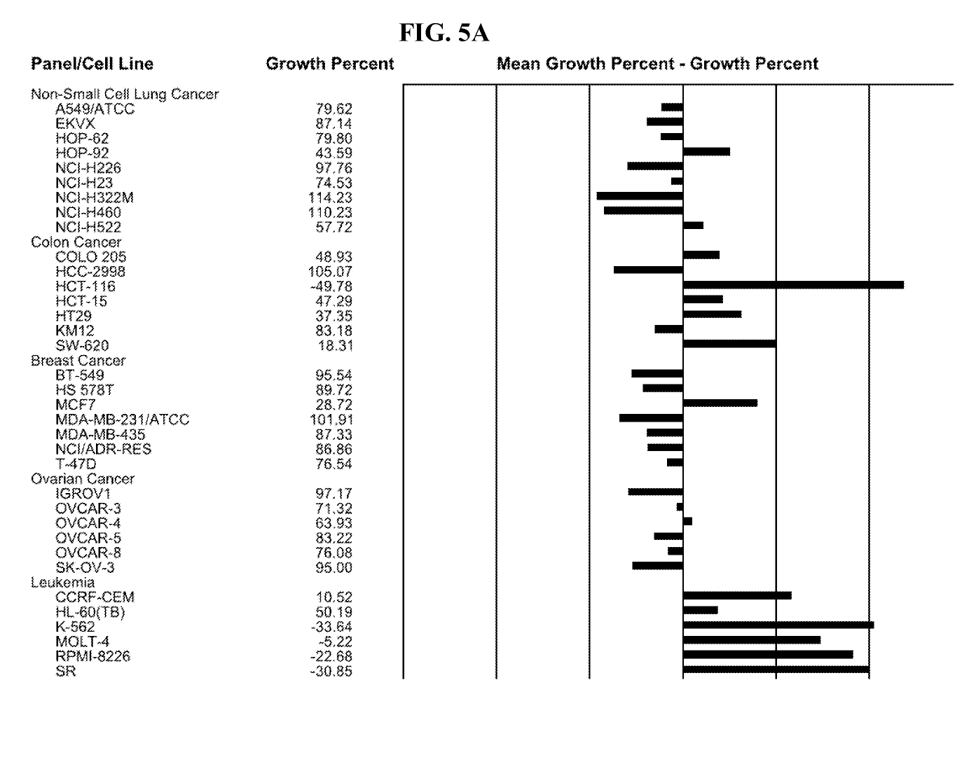

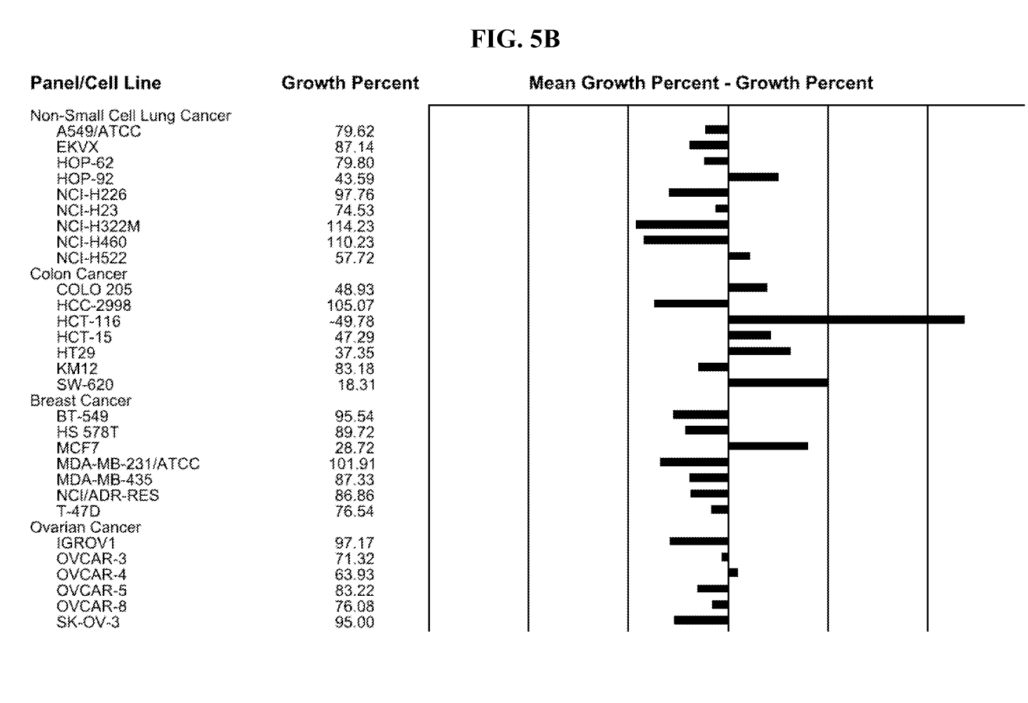

Results from the dose response screen for compound LDN-192960 are shown in FIGS. 1, 2A-2B, 3A-3B, and 4A-4B. Additionally, the results of a preliminary single dose experiment are shown in FIG. 5A and 5B.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula I: 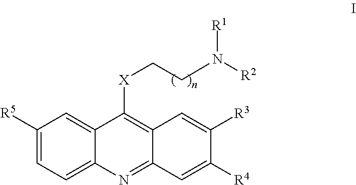
or pharmaceutically acceptable salt thereof, wherein:
X is CH₂, S, or NR^{A};
R¹ and R² are each independently H, C₁₋₆alkyl, —C(O)R^{A}, —C(O)OR^{A}, or —C(O)NR^{A}R^{B};
or R¹ and R² together with the N atom to which they are attached form a heterocyclic group selected from: a phthalimide group, a benzo[d]isothiazol-3(2H)-one 1,1-dioxide, a benzo[d][1,3,2]dithiazole 1,1,3,3-tetraoxide, a 3-iminoisoindolin-1-one, and an isoindoline-1,3-diimine; wherein the phthalamide group is optionally substituted by halo, OH, C₁₋₆haloalkyl, C₁₋₆alkoxy, C₁₋₆haloalkoxy, —CN, —C(O)NR^{A}R^{B}, —S(O)₂R^{A}, —S(O)₂NR^{A}R^{B}, or —NR^{A}R^{B};
R³, R⁴ and R⁵ are each independently H, halo, OH, C₁₋₆haloalkyl, C₁₋₆alkoxy, C₁₋₆haloalkoxy, —CN, —C(O)NR^{A}R^{B}, —S(O)₂R^{A}, —S(O)₂NR^{A}R^{B}, or —NR^{A}R^{B};
R^{A} and R^{B} are each independently H or C₁₋₆alkyl; and
n is 1, 2, 3, 4, or 5.

2. The compound of claim 1, or pharmaceutically acceptable salt thereof, wherein X is S or CH₂.

3. The compound of claim 1, or pharmaceutically acceptable salt thereof, wherein R¹ and R² are each independently H, C₁₋₆alkyl, or R¹ and R² together with the N atom to which they are attached form a phthalimide group, optionally substituted by halo, OH, C₁₋₆haloalkyl, C₁₋₆alkoxy, C₁₋₆haloalkoxy, —CN, —C(O)NR^{A}R^{B}, —S(O)₂R^{A}, —S(O)₂NR^{A}R^{B}, or —NR^{A}R^{B}.

4. The compound of claim 1, or pharmaceutically acceptable salt thereof, wherein R¹ and R² together with the N atom to which they are attached form an unsubstituted phthalimide group.

5. The compound of claim 1, or pharmaceutically acceptable salt thereof, wherein X is S, and R¹ and R² are both H.

6. The compound of claim 1, or pharmaceutically acceptable salt thereof, wherein R³ and R⁵ are both C₁₋₆alkoxy.

7. The compound of claim 1, or pharmaceutically acceptable salt thereof, wherein R⁴ is halo.

8. The compound of claim 1, or pharmaceutically acceptable salt thereof, wherein R⁴ is chloro.

9. The compound of claim 1, or pharmaceutically acceptable salt thereof, wherein n is 2.

10. The compound of claim 1, or pharmaceutically acceptable salt thereof, wherein:
X is S or CH₂;
R¹ and R² are each H;
R³ and R⁵ are each independently H, OH, methyl, methoxy, or chloro; and
n is 2 or 3.

11. The compound of claim 1, or pharmaceutically acceptable salt thereof, wherein:
X is S;
R¹ and R² are each independently H or methyl;
R³ and R⁵ are each independently H or methoxy; and
n is 2 or 3.

12. The compound of claim 1, or pharmaceutically acceptable salt thereof, wherein:
X is S;
R¹ and R² are each H, or R¹ and R² together with the N atom to which they are attached form an unsubstituted phthalimide group;
R³ is methoxy;
R⁴ is H;
R⁵ is methoxy; and
n is 2.

13. The compound of claim 1, selected from:
3 -((2,7-dimethoxyacridin-9-yl)thio)propan-1-amine;
3-((2-methoxyacridin-9-yl)thio)propan-1-amine;
3-((2,3-dimethoxyacridin-9-yl)thio)propan-1-amine;
9-((3-aminopropyl)thio)acridine-2,7-diol;
N¹-(2,4-dimethoxyacridin-9-yl)-N³,N³-dimethylpropane-1,3-diamine;
N¹-(2,4-dimethoxyacridin-9-yl)-N³,N³-diethylpropane-1,3-diamine;
3-((2-methoxy-7-methylacridin-9-yl)thio)propan-1-amine;
3-((2-chloro-7-methoxyacridin-9-yl)thio)propan-1-amine;
3-((3-chloro-2-methoxyacridin-9-yl)thio)propan-1-amine;
2-((2,7-dimethoxyacridin-9-yl)thio)ethanamine;
4-((2,7-dimethoxyacridin-9-yl)thio)butan-1-amine;
3-((2,7-dimethoxyacridin-9-yl)thio)-N-methylpropan-1-amine;
3-(acridin-9-ylthio)propan-1-amine;
N¹-(2,7-dimethoxyacridin-9-yl)propane-1,3-diamine;
3-((2,7-dimethoxyacridin-9-yl)oxy)propan-1-amine;
4-(2,7-dimethoxyacridin-9-yl)butan-1-amine;
3-((7-methoxy-1,2,3,4-tetrahydroacridin-9-yl)thio)propan-1-amine;
2-(3-((2,7-dimethoxyacridin-9-yl)thio)propyl)isoindoline-1,3-dione;
2-(3-((2-methoxyacridin-9-yl)thio)propyl)isoindoline-1,3-dione; and
3-((2,7-dimethoxyacridin-9-yl)thio)-N,N-dimethylpropan-1-amine, or pharmaceutically acceptable salt thereof.

14. The compound of claim 1, wherein the compound is:
3-((2,7-dimethoxyacridin-9-yl)thio)propan-1-amine, or pharmaceutically acceptable salt thereof.

15. A composition comprising a compound of claim 1, and a pharmaceutically acceptable carrier, or pharmaceutically acceptable salt thereof.

16. A method for treating a disease in a subject, the method comprising administering to said subject in need of such treatment a therapeutically effective amount of a compound according to claim 1, or pharmaceutically acceptable salt thereof.

17. The method of claim 16, wherein said disease is cancer, Down's Syndrome, diabetes, cardiac ischemia, Alzheimer's Disease, or anemia.

18. The method claim 17, wherein said disease is cancer

19. The method of claim 18, wherein said cancer is a hematological malignancy.

20. The method of claim 19, wherein said hematological malignancy leukemia or lymphoma.

21. The method of claim 16, wherein said subject is a mammal.

22. The method of claim 21, wherein said mammal is a human.
